(19) United States
(12) Patent Application Publication
(10) Pub. No.: US 2025/0387560 A1
(43) Pub. Date: Dec. 25, 2025

(54) Systems, Methods, and Apparatuses for Sensing and Fluid Delivery and Retention of Related Components

(71) Applicant: DEKA Products Limited Partnership, Manchester, NH (US)

(72) Inventors: Dean Kamen, Bedford, NH (US); Kevin L. Grant, Litchfield, NH (US); Tyler G. Caron, Manchester, NH (US); Brian D. Tracey, Litchfield, NH (US)

(21) Appl. No.: 18/882,023

(22) Filed: Sep. 11, 2024

Related U.S. Application Data
(60) Provisional application No. 63/582,223, filed on Sep. 12, 2023.
(60) Provisional application No. 63/661,937, filed on Jun. 20, 2024.
(60) Provisional application No. 63/673,352, filed on Jul. 19, 2024.
(60) Provisional application No. 63/683,474, filed on Aug. 15, 2024.

(51) Int. Cl. A61M 5/142; A61M 5/14; A61M 5/168

(52) U.S. Cl. CPC A61M 5/14248; A61M 5/1413; A61M 5/16831; A61M 2005/14268; A61M 2205/0266; A61M 2209/088

(57) ABSTRACT

A retention assembly for an ambulatory infusion pump assembly may comprise an adhesive pad including a central aperture and at least one protruding portion extending from a periphery of the adhesive pad. The retention assembly may further comprise a socket coupled to the adhesive pad. The socket may comprise a base portion with a socket aperture aligned with the central aperture. The socket may further comprise a wall extending from a periphery of the base portion. An inner surface of the wall may have a recess defined therein. The socket may further comprise at least one clip. The socket may further comprise at least one alignment projection extending from the base portion. The socket may further comprise at least one air channel recessed into the base plate extending from socket aperture to the periphery of the base portion.

### CROSS-REFERENCE TO RELATED APPLICATIONS

The present application claims the benefit of U.S. Provisional Application Ser. No. 63/582,223 filed Sep. 12, 2023 and entitled Systems, Methods, and Apparatuses, for Sensing and Fluid Delivery (Attorney Docket No. 00101.00361.AB159), and claims the benefit of U.S. Provisional Application Ser. No. 63/661,937 filed Jun. 20, 2024 and entitled Systems, Methods, and Apparatuses, for Sensing and Fluid Delivery (Attorney Docket No. 00101.00414.AB446) and claims the benefit of U.S. Provisional Application Ser. No. 63/673,352 filed Jul. 19, 2024 and entitled Systems, Methods, and Apparatuses, for Sensing and Fluid Delivery (Attorney Docket No. 00101.00432.AB558) and claims the benefit of U.S. Provisional Application Ser. No. 63/683,474 filed Aug. 15, 2024 and entitled Systems, Methods, and Apparatuses, for Sensing and Fluid Delivery (Attorney Docket No. 00101.00435.AB565) which are each hereby incorporated herein by reference in their entireties.

### BACKGROUND

### Field of Disclosure

This disclosure relates to fluid infusion. More specifically, this disclosure relates to fluid infusion device assemblies.

### Description of Related Art

Many potentially valuable medicines or compounds, including biologicals, are not orally active due to poor absorption, hepatic metabolism or other pharmacokinetic factors. Additionally, some therapeutic compounds, although they can be orally absorbed, are sometimes required to be administered so often it is difficult for a patient to maintain the desired schedule. In these cases, parenteral delivery is often employed or could be employed. Other medicines can be administered by routes other than parenteral, but the bioavailability of the drug varies from an ideal amount over time.

Effective parenteral routes of drug delivery, as well as other fluids and compounds, such as subcutaneous injection, intramuscular injection, and intravenous (IV) administration include puncture of the skin with a needle or stylet. Insulin is an example of a therapeutic fluid that is self-injected by millions of diabetic patients. Users of parenterally delivered drugs would benefit from a wearable device that would automatically deliver needed drugs/compounds over a period of time.

To this end, there have been efforts to design portable devices for the controlled release of therapeutics. Such devices are known to have a reservoir such as a cartridge, syringe, or bag, and to be electronically controlled. These devices suffer from a number of drawbacks including the malfunction rate. Reducing the size, weight and cost of these devices is also an ongoing challenge.

### SUMMARY

In accordance with an exemplary embodiment of the present disclosure a retention assembly for an ambulatory infusion pump assembly may comprise an adhesive pad. The adhesive pad may include a central aperture. The adhesive pad may also include at least one protruding portion extending from a periphery of the adhesive pad. The retention assembly may further comprise a socket coupled to the adhesive pad. The socket may comprise a base portion. The base portion may include a socket aperture aligned with the central aperture. The socket may further comprise a wall extending from a periphery of the base portion. The wall may include an inner surface with a recess defined therein. The socket may further comprise at least one clip. The socket may further comprise at least one alignment projection extending from the base portion. The socket may further comprise at least one air channel recessed into the base plate extending from socket aperture to the periphery of the base portion.

In accordance with another exemplary embodiment of the present disclosure a retention assembly for an ambulatory infusion pump assembly may comprise an adhesive bearing pad including a main region. The pad may include a pad aperture centrally disposed in the main region. The pad may further include at least one protrusion extending from the periphery of the main region. The retention assembly may further comprise a socket coupled to the adhesive pad. The socket may comprise a base portion. The base portion may include a centrally disposed socket aperture aligned with the pad aperture. The socket may further comprise a wall extending from a periphery of the base portion. The wall may have at least one ledge extending from a portion of the wall most distal the base portion. The socket may further comprise an appendage projecting from the base portion. The socket may further comprise at least one clip. The socket may further comprise at least one pump assembly alignment receptacle recessed into at least the wall. The socket further comprise at least one alignment projection extending from the base plate. The socket may further comprise at least one air flow channel recessed into a face of the base plate and extending from the periphery of the base portion to the socket aperture.

In some embodiments, the at least one protrusion may include a plurality of spoke members which extend outwardly from the periphery of the main region. In some embodiments, the main region may be round and the spoke members may each have a length equal to at least 50% of a radius defining the periphery of the main region. In some embodiments, the socket includes a rigid portion and a complaint portion. In some embodiments, the compliant portion may be overmolded onto the rigid portion. In some embodiments, the base portion may be defined in the rigid portion and the complaint portion may include a flange which at least partially surrounds the base portion. The flange may be proud of the base portion and including an innermost section which tapers toward a surface of the base portion. In some embodiments, the at least one alignment projection may include at least one tooth projection on the appendage of the socket. In some embodiments, the at least one alignment projection may be tapered such that its cross-sectional area decreases as distance from the base plate increase. In some embodiments, the wall may be present along a minority of the periphery of the base plate. In some embodiments, the wall may include a ramped exterior surface. In some embodiments, at least one of the at least one alignment projection may be configured to mate into a pocket defined in a tab extending from a peripheral wall of a reservoir assembly of the infusion pump assembly. In some embodiments, at least one of the at least one alignment projection may be configured to mate into a well defined in a bottom face of a reservoir assembly of the infusion pump assembly. In some embodiments, at least one of the at least one alignment projection may be configured to mate into a recess formed in a transition between a peripheral sidewall of a reservoir assembly of the infusion pump assembly and a bottom face of the reservoir assembly. In some embodiments, at least one of the at least one alignment projection may be configured to mate into a recess defined adjacent the periphery of a bottom face of a reservoir assembly of the infusion pump assembly. In some embodiments, the at least one alignment projection and the alignment receptacle may be arranged in a poka-yoke configuration.

In accordance with yet another embodiment of the present disclosure a method of removably coupling an ambulatory infusion pump assembly to a patient may comprise adhering a socket to the patient. The method may further comprise aligning the infusion pump with at least one alignment projection and at least one alignment receptacle defined in the socket. The method may further comprise deflecting at least one clip and deflecting a wall having at least one ledge defined thereon from respective resting states to respective deflected state by pressing the infusion pump assembly toward a base plate of the socket. The method may further comprise advancing the infusion pump assembly against the base plate and restoring the clip and the wall to their respective resting states. The method may further comprise capturing the infusion pump assembly with the clip and the at least one ledge defined on the wall.

In some embodiments, the method may further comprise seating a raised node of a reservoir assembly of the infusion pump assembly into the alignment receptacle. In some embodiments, method further may comprise mating each of the at least one alignment projection into a respective recess defined in a surface of a reservoir assembly of the infusion pump assembly. In some embodiments, adhering the socket to the patient may comprise placing an adhesive pad including a central aperture against the patient and wrapping at least one spoke member extending from a periphery of a main region of the adhesive pad around at least one contour of the patient's body.

In accordance with another example embodiment of the present disclosure an example dispensing assembly may comprise a first housing portion. The first housing portion may comprise a power source. The first housing portion may further comprise a mechanical actuation assembly. The first housing portion may further comprise a controller configured to orchestrate actuation of the mechanical actuation assembly. The first housing portion may further comprise a transmitter. The dispensing assembly may further comprise a second housing portion releasably coupled to the first housing portion. The second housing portion may comprise a reservoir assembly in fluid communication with a set of tubing. The second housing portion may further comprise at least a pair of sensing micropenetrators in communication with respective conductive traces. The conductive traces may establish electrical communication with cooperative conductive pathways in the first housing portion when the second housing portion is driven to a coupled state with respect to the first housing portion.

In some embodiments, the pair of micropenetrators may extend proud of an exterior surface of the second housing portion and may be coupled thereto in during an injection molding operation. In some embodiments, the pair of micropenetrators may extend proud of an exterior surface of the second housing portion and may be coupled thereto via adhesive. In some embodiments, the controller may be configured to analyze data signals from the pair of micropenetrators and determine an analyte level based upon the data signals. In some embodiments, the sensing micropenetrators may be coupled to a carriage. The carriage may be disposed in a guide channel of the second housing portion. In some embodiments, the reusable housing assembly may further comprise a plunger. The plunger may be associated with a bias member which urges the plunger in a first direction. In some embodiments, the plunger may align with the guide channel when the first and second housing portions are in a coupled state. The first direction may be a direction toward a direction of extension of the micropenetrators from the carriage. In some embodiments, the sensing micropenetrators may be constructed of silicon. In some embodiments, the sensing micropenetrators may be configured to sense blood glucose. In some embodiments, the reservoir may be filled with insulin. In some embodiments, the reservoir may be filled with glucagon. In some embodiments, the reservoir may be filled with a regulatory hormone. In some embodiments, the reservoir may be filled with a peptide based medicament.

In accordance with another embodiment of the present disclosure an example retention assembly for an ambulatory infusion pump assembly may comprise an adhesive bearing pad including a main region and at least one protrusion extending from the periphery of the main region. The retention assembly may further comprise a socket coupled to the adhesive pad. The socket may comprise a base portion. The socket may further comprise a wall extending from a periphery of the base portion. The socket may further comprise an appendage projecting from the base portion. The socket may further comprise at least one dispensing assembly alignment receptacle recessed into at least the wall. The socket may further comprise at least one alignment projection extending from the base plate. The socket may further comprise at least one magnet coupled to the base portion.

In some embodiments, the socket further may comprise a centrally disposed socket aperture. In some embodiments, the adhesive bearing pad may include a pad aperture aligned with the socket aperture. In some embodiments, the at least one magnet may include a plurality of magnets. In some embodiments, each of the at least one magnet may be a rare earth magnet. In some embodiments, the socket may further comprise a clip. In some embodiments, the at least one protrusion may include a plurality of members which extend outwardly from the periphery of the main region. In some embodiments, the socket may include a rigid portion and a complaint portion. In some embodiments, the compliant portion may be overmolded onto the rigid portion. In some embodiments, the at least one alignment projection may include at least one tooth projection on the appendage of the socket. In some embodiments, the at least one alignment projection may be tapered such that its cross-sectional area decreases as distance from the base plate increases. In some embodiments, the wall may be present along a minority of the periphery of the base plate.

In accordance with another embodiment of the present disclosure, an exemplary retention assembly for an ambulatory infusion pump assembly may comprise an adhesive bearing pad including a main region and at least one protrusion extending from the periphery of the main region. The retention assembly may further comprise a socket coupled to the adhesive pad. The socket may comprise a base portion. The socket may further comprise a wall extending from a periphery of the base portion. The socket may further comprise an appendage projecting from the base portion. The socket may further comprise at least one dispensing assembly alignment receptacle recessed into at least the wall. The socket may further comprise at least one alignment projection extending from the base plate. The socket may further comprise at least one ferromagnetic body coupled to the base portion.

In some embodiments, the socket may further comprise a centrally disposed socket aperture. In some embodiments, the adhesive bearing pad may include a pad aperture aligned with the socket aperture. In some embodiments, the at least one magnet may include a plurality of magnets. In some embodiments, each of the at least one magnet may be a rare earth magnet. In some embodiments, the socket further may comprise a clip. In some embodiments, the at least one protrusion includes a plurality of members which extend outwardly from the periphery of the main region. In some embodiments, the socket may include a rigid portion and a complaint portion. In some embodiments, the compliant portion may be overmolded onto the rigid portion. In some embodiments, the at least one alignment projection may include at least one tooth projection on the appendage of the socket. In some embodiments, the at least one alignment projection may be tapered such that its cross-sectional area decreases as distance from the base plate increases. In some embodiments, the wall may be present along a minority of the periphery of the base plate.

In accordance with yet another embodiment of the present disclosure, an example adhesive assembly for retention of an ambulatory infusion pump to a patient may comprise an adhesive bearing pad including a main region and a number of protrusions extending outwardly from the periphery of the main region. The adhesive bearing pad may include an adhesive bearing first face and an opposing face. The adhesive assembly may further comprise at least one magnetic puck, each of the at least one magnetic puck may include a magnet and a puck housing. Each of the at least one magnetic puck may be sonically welded to the opposing face of the adhesive bearing pad.

In accordance with another example embodiment of the present disclosure an example ambulatory infusion system may comprise an infusion pump assembly. The infusion pump assembly may comprise a first portion having a controller, a transmitter, and a mechanical actuation assembly. The infusion pump assembly may further comprise a second portion including a reservoir and a fluid path extending from the reservoir toward a delivery outlet. The infusion pump assembly may further comprise a securement body at least partially formed from a material selected from a list consisting of a ferromagnetic material and permanently magnetized material. The infusion pump assembly may further comprise a retainer body separate from the infusion pump including at least one magnet configured to magnetically hold the securement body in place when a gap of at least one millimeter which is filled with garment material is present between the retainer body and securement body.

In some embodiments, the mechanical actuation assembly may include no motor. In some embodiments, the mechanical actuation assembly may include at least one shape memory actuator. In some embodiments, the mechanical actuation assembly may include at least one wire formed of a shape memory alloy. In some embodiments, the fluid path may be covered in at least one flexible membrane and the fluid path may define at least one pumping chamber and at least one valve station. In some embodiments, the mechanical actuation assembly may include a plunger for each of the at least one pumping chamber and a valve actuator for each of the at least one valve station. In some embodiments, the securement body may be attached to an exterior of the second portion of the infusion pump assembly via an adhesive. In some embodiments, the first portion may further comprise an acoustic volume sensor assembly and the fluid path may include a sensing chamber having a variable volume. In some embodiments, the system may further comprise a run of infusion tubing extending from the second portion of the infusion pump assembly to an infusion device including a cannula. In some embodiments, the securement body may be disposed within a recess in an exterior surface of the second portion of the infusion pump assembly.

In accordance with another example embodiment of the present disclosure, a retention assembly for an ambulatory infusion pump may comprise an adhesive bearing pad including a main region and a number of petal shaped protrusions extending from the periphery of the main region and being spaced about the main region at regular angular intervals. The retention assembly may further comprise a socket coupled to the adhesive pad. The socket may comprise a base portion with a centrally disposed socket aperture. The socket may further comprise a wall extending from a periphery of the base portion and having at least one wall ledge extending from a portion of the wall most distal the base portion. The wall interrupted by a respective fenestration aligned with each of the at least one wall ledge. The socket may further comprise an appendage projecting from the base portion. The socket may further comprise a clip at an end of the appendage most distal to the base portion. The clip may have a raised wall with at least one clip ledge extending from the raised wall. The clip ledge may be positioned intermediate the appendage and the end of the raised wall most distal the appendage. There may be a respective passage through the appendage associated with each clip ledge. Each clip ledge may extend over the respective passage. The socket may further comprise at least one alignment projection extending from the appendage.

In some embodiments, the socket may further comprise a centrally disposed socket aperture. In some embodiments, the adhesive bearing pad may include a pad aperture aligned with the socket aperture. In some embodiments, the base portion may include at least one magnet. In some embodiments, the base portion may include at least one retention post. In some embodiments, the base portion may further comprise at least one retention post clutch. In some embodiments, the retention assembly may further comprise a release liner having a peripheral protuberance. In some embodiments, the base portion and appendage may be formed of rigid material. In some embodiments, the socket may further comprise a compliant portion overmolded onto at least the base portion. In some embodiments, the at least one alignment projection may include at least one tooth projection. In some embodiments, the at least one alignment projection may be tapered such that its cross-sectional area decreases as distance from the base portion increases. In some embodiments, the wall may be present along a minority of the periphery of the base portion. In some embodiments, the wall may be present along no more than a third of the periphery of the base portion. In some embodiments, the adhesive bearing pad may be coupled to the socket by one of a heat stake and a sonic weld. In some embodiments, the socket may include at least one air flow channel recessed into a face of the base portion. In some embodiments, the base portion may include a least one pass-through extending therethrough. Each of the at least one pass-through may be aligned with a respective aperture in the adhesive pad.

In accordance with another example embodiment of the present disclosure a retention assembly for an ambulatory infusion pump assembly may comprise an adhesive bearing pad including a main region and at least one protrusion extending from the periphery of the main region. The retention assembly may further comprise a socket coupled to the adhesive pad. The socket may comprise a base portion. The socket may further comprise a wall extending from a periphery of the base portion. The socket may further comprise an appendage projecting from the base portion. The socket may further comprise at least one dispensing assembly alignment receptacle recessed into at least the wall. The socket may further comprise at least one alignment projection extending from the base plate. The socket may further comprise at least infusion pump mounting component selected from a list consisting a retention post and a retention post clutch coupled to the base portion.

In some embodiments, the socket may further comprise a centrally disposed socket aperture. In some embodiments, the adhesive bearing pad may include a pad aperture. In some embodiments, the socket may further comprise a clip. In some embodiments, the at least one protrusion may include a plurality petal members spaced at regular angular intervals about the main region. In some embodiments, the socket may include a rigid portion and a complaint portion. In some embodiments, the compliant portion may be overmolded onto the rigid portion. In some embodiments, the at least one alignment projection may include at least one tooth projection on the appendage of the socket. In some embodiments, the at least one alignment projection may be tapered such that its cross-sectional area decreases as distance from the base plate increases. In some embodiments, the wall may be present along a minority of the periphery of the base plate.

In accordance with yet another example embodiment of the present disclosure a reservoir assembly for an ambulatory infusion pump may comprise a first unit. The first unit may comprise a reservoir with a main interior volume. The first unit may further comprise a septum having a face in fluid communication with the main interior volume. The first unit may further comprise a pump chamber, set of valve chambers, and a variable volume sensing chamber in fluid communication with each other and the main interior volume via a set of flow paths. The first unit may further comprise at least one flexible membrane covering the pump chamber, set of valve chambers, and the variable volume sensing chamber. The first unit may further comprise a length of tubing coupled to an outlet of the set of flow paths. The reservoir assembly may further comprise a shell unit including a tab and at least one shell recess. The first unit may be coupled within the shell unit. The reservoir assembly may further comprise at least one ambulatory mounting component selected from a list consisting of at least one magnet, at least one ferromagnetic body, at least one retention post, and at least one retention post clutch.

In some embodiments, at least one of the at least one membrane may partially form a portion of the set of flow paths. In some embodiments, the at least one ambulatory mounting component may be coupled to the shell unit. In some embodiments, each of at least one ambulatory mounting component may be coupled within a respective receptacle defined in the shell. In some embodiments, each of the at least ambulatory mounting component may be coupled within the respective receptacle in the shell in a manner selected from a list consisting of the following, adhesive, overmolding of the shell to the at least one ambulatory mounting component, and swaging of the shell over at portion of each of the at least one ambulatory mounting component. In some embodiments, the at least one ambulatory mounting component may be coupled to the first unit. In some embodiments, the at least one ambulatory mounting component may be coupled to the first unit and extends through the shell. In some embodiments, the shell may include a passage aligned with the septum. In some embodiments, the main interior volume may be defined, at least partially, by a flexible wall. In some embodiments, the main interior volume may have a filled state and a depleted state. The main interior volume may hold at least 3 ml of fluid in the filled state.

### BRIEF DESCRIPTION OF THE DRAWINGS

These and other aspects will become more apparent from the following detailed description of the various embodiments of the present disclosure with reference to the drawings wherein:

FIG. 1 depicts a block diagram of an example drug delivery system;

FIGS. 2-3E depict block diagram views of example actuation assemblies which may be included in certain example dispensing assemblies;

FIGS. 4A-B depict perspective views of an example dispensing assembly;

FIGS. 5A-B depict exploded views of an exemplary dispensing assembly;

FIGS. 6A-C depict a number of bottom plan views of various example reservoir assemblies;

FIG. 7 depicts a plan view of an example socket for a dispensing assembly;

FIG. 8 depicts a side view of an example socket for a dispensing assembly;

FIG. 9 depicts a side view of an example dispensing assembly installed in an example socket;

FIGS. 10-12 depict perspective views of exemplary sockets;

FIG. 13 depicts a perspective view of an example reservoir assembly retained within an example socket;

FIG. 14 depicts a cross-sectional view of an example reservoir assembly retained within an example socket;

FIG. 15 depicts a plan view of an example adhesive pad;

FIG. 16 depicts a plan view of another example adhesive pad;

FIG. 17 depicts a plan view of another example adhesive pad;

FIG. 18A depicts a plan view of an example adhesive assembly;

FIG. 18B depicts an exploded view of an example adhesive assembly;

FIG. 19 depicts a perspective view of an example adhesive assembly and example socket;

FIG. 20 depicts a perspective view of an example socket;

FIG. 21 depicts a perspective view of an example adhesive assembly and example socket;

FIG. 22 depicts a plan view of an exemplary compliant portion which may optionally be included as part of a socket;

FIG. 23 depicts a plan view of an exemplary compliant portion which may optionally be included as part of a socket;

FIG. 24 depicts a plan view of another example compliant portion which may optionally be included as part of a socket;

FIG. 25 depicts a perspective view of an example socket including a compliant portion which is overmolded onto a rigid portion of the socket;

FIG. 26A depicts a plan view of an example socket which may be particularly well suited for athletic activities;

FIG. 26B depicts a side view of an example socket which may be particularly well suited for athletic activities;

FIG. 27A depicts a perspective view of an example socket which may be particularly well suited for athletic activities;

FIG. 27B depicts a bottom plan view of an example socket which may be particularly well suited for athletic activities;

FIG. 28A depicts a perspective view of an example socket which may be particularly well suited for athletic activities;

FIG. 28B depicts a bottom plan view of an example socket which may be particularly well suited for athletic activities;

FIGS. 29A-30B depict various example retention assemblies and example reservoir assemblies which may be coupled via a magnetic arrangement;

FIG. 31 depicts an example socket and example adhesive pad with a magnetic coupling arrangement;

FIG. 32 depicts an example adhesive pad with a plurality of exemplary magnetic pucks coupled thereto;

FIG. 33A depicts an example retainer for a dispensing assembly;

FIGS. 33B-33C depict example dispensing assemblies and example retainers;

FIG. 34A depicts an example dispensing assembly including an example reservoir assembly including a number of example posts engaged with clutches to hold the reservoir assembly in place on a garment;

FIG. 34B depicts an example dispensing assembly including an example reservoir assembly having a number of clutches engaged with example posts extending from an example retainer to hold the reservoir assembly in place on a garment;

FIG. 35 depicts an example dispensing assembly include a reservoir assembly having a safety pin coupled thereto;

FIG. 36 depicts a block diagram of a drug delivery system including a dispensing assembly having at least delivery sharp type access member;

FIG. 37 depicts a block diagram of a drug delivery system including a dispensing assembly having at least one sensing type access member;

FIG. 38 depicts a block diagram of a drug delivery system including a dispensing assembly having at least one delivery sharp type access member and at least one sensing type access member;

FIG. 39A depicts a bottom perspective view of an example dispensing assembly including a number of access members;

FIG. 39B depicts an exploded view of the dispensing assembly of FIG. 39A;

FIG. 40 depicts an example embodiment of a reservoir assembly which may be included in various dispensing assemblies described herein;

FIG. 41 depicts an example embodiment of a reservoir assembly which may be included in various dispensing assemblies described herein;

FIG. 42 depicts an alternative embodiment of the actuation assembly of FIG. 3A;

FIG. 43A depicts an exemplary embodiment of a microneedle; and

FIG. 43B depicts an exemplary embodiment of a sensing micropenetrator.

### DETAILED DESCRIPTION

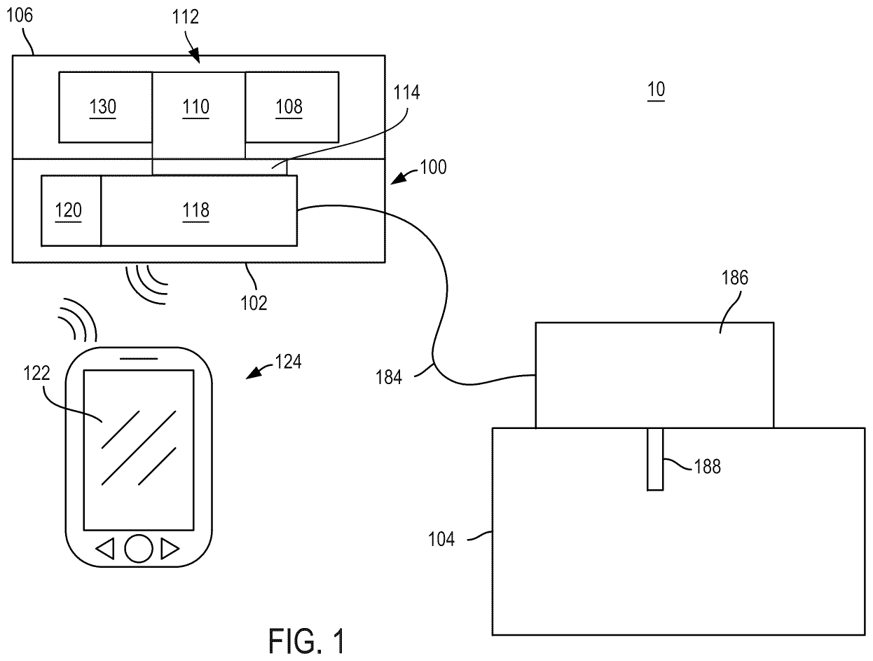

Referring to FIG. 1, an exemplary drug delivery system 10 is shown. The example drug delivery system 10 includes a dispensing assembly 100 which may be formed of a reservoir assembly 102 and a reusable housing assembly 106. The reservoir assembly 102 may include a container or reservoir 118 and an access 120 (e.g. a pierceable member such as a septum). The reservoir assembly 102 may be releasably coupled to the reusable housing assembly 106. In certain examples, the reservoir assembly 102 may include multiple reservoirs 118 which may each contain different agents. The dispensing assembly 100 may include an actuation assembly 112. The reusable housing assembly 106 may include a controller 108 and a mechanical actuation assembly 110 which may be controlled to selectively dispense fluid from the reservoir assembly 102. In certain embodiments, the mechanical actuation assembly 110 may act on a fluid contacting portion 114 of the actuation assembly 112 included in the reservoir assembly 102. The fluid contacting portion 114 may include valving and pumping components which may come into direct contact with agent from the reservoir(s) 118 and be operated by the mechanical actuation assembly 110.

As shown, a dispensing assembly 100 may be used in conjunction with an infusion device 186 of a drug delivery system 10. The infusion device 186 may be configured to be inserted into a patient to provide a fluid pathway from the reservoir assembly 102 into the patient 104 (e.g. to a subcutaneous layer of the patient's 104 skin). To facilitate establishment of a fluid pathway into the patient's 104 skin, the infusion device 186 may include a needle or cannula 188. The infusion device 186 may be fluidly connected to a length of tubing 184 and/or to an infusion pump or dispensing assembly 100. An outlet of the reservoir 118 may couple the reservoir assembly 102 directly or indirectly to the tubing 184 or infusion device 186 in certain embodiments.

The dispensing assembly 100 may be controlled by a remote device 124 through wireless communication with the controller 108 (though wired communication is also possible). The remote device 124 may be dedicated to communication with the dispensing assembly 100, or may be a more general device such as a cell phone or tablet running a specific application for the drug delivery system 10. The remote device 124 may have a display 122 for conveying information from and/or related to the dispensing assembly 100, including status, warnings, alarms, etc. The remote device 124 may also be used to enter information for relay to the dispensing assembly 100, specifying infusion programs and initiating or transitioning between operations, functions, and modes of the dispensing assembly 100. The dispensing assembly 100 may include a transmitter 130 (which may send and receive data) for establishing wireless communication with other components of the system 10.

The various components described in relation to FIG. 1 may be, but are not limited to, those shown and described in one or more of the following: U.S. patent application Ser. No. 13/788,260, filed Mar. 7, 2013 and entitled Infusion Pump Assembly, now U.S. Publication No. US-2014-0107579, published Apr. 17, 2014 (Attorney Docket No. K40); U.S. Pat. No. 8,491,570, issued Jul. 23, 2013 and entitled Infusion Pump Assembly (Attorney Docket No. G75); U.S. Pat. No. 8,414,522, issued Apr. 9, 2013 and entitled Fluid Delivery Systems and Methods (Attorney Docket No. E70); U.S. Pat. No. 8,262,616, issued Sep. 11, 2012 and entitled Infusion Pump Assembly (Attorney Docket No. F51); U.S. Pat. No. 7,306,578, issued Dec. 11, 2007 and entitled Loading Mechanism for Infusion Pump (Attorney Docket No. C54); U.S. Provisional Application No. 62/597,246, filed Dec. 11, 2017 and entitled Infusion Pump Assembly (Attorney Docket No. P51); U.S Publication No. 2015/0281863, published Oct. 5, 2017 and entitled Infusion Set and Inserter Assembly (Attorney Docket No. U64); U.S. application Ser. No. 15/961,238, filed Apr. 24, 2018 and entitled Apparatus, System and Method for Fluid Delivery (Attorney Docket No. X37); U.S. Pat. No. 9,617,020, issued Apr. 11, 2017 and entitled Apparatus, System and Method for Fluid Delivery (Attorney Docket No. M60); U.S. patent application Ser. No. 18/550,497, filed Sep. 14, 2023 and entitled Infusion Device Assembly (Attorney Docket No. AA813),; and US Publication No. 2023/0277759, filed Mar. 3, 2023, and entitled Systems, Methods, and Apparatuses for Medical Agent Administration (Attorney Docket No. 00101.00359.AB108); U.S. Publication No. 2024/0066217, filed Aug. 24, 2023, entitled Infusion Set and Inserter Assembly Apparatuses, Systems, and Methods (Attorney Docket No. 00101.00365.AB184), all of which are hereby incorporated herein by reference in their entireties. The systems and methods (including the disposable housing assemblies, reservoirs, filling aids, charging systems, volume sensing arrangements, control systems, inserter assemblies, etc.) described in any of the above-referenced applications and patents may also be used in conjunction with the various embodiments shown and described herein. The embodiments shown and described herein are not, however, limited to use therewith.

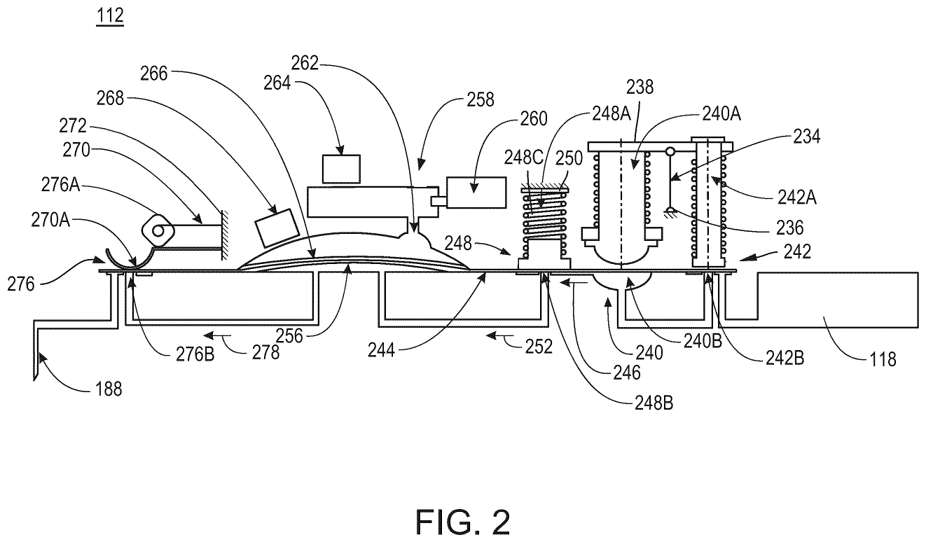

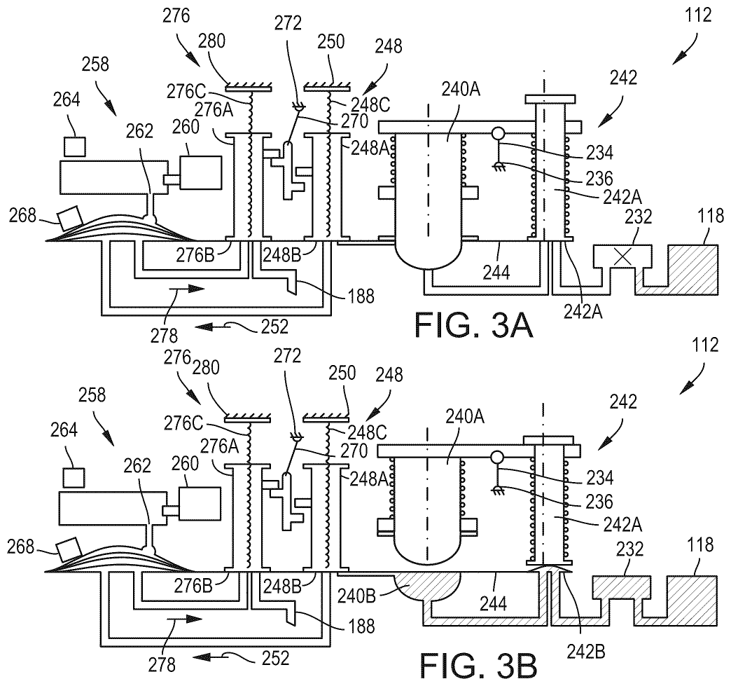

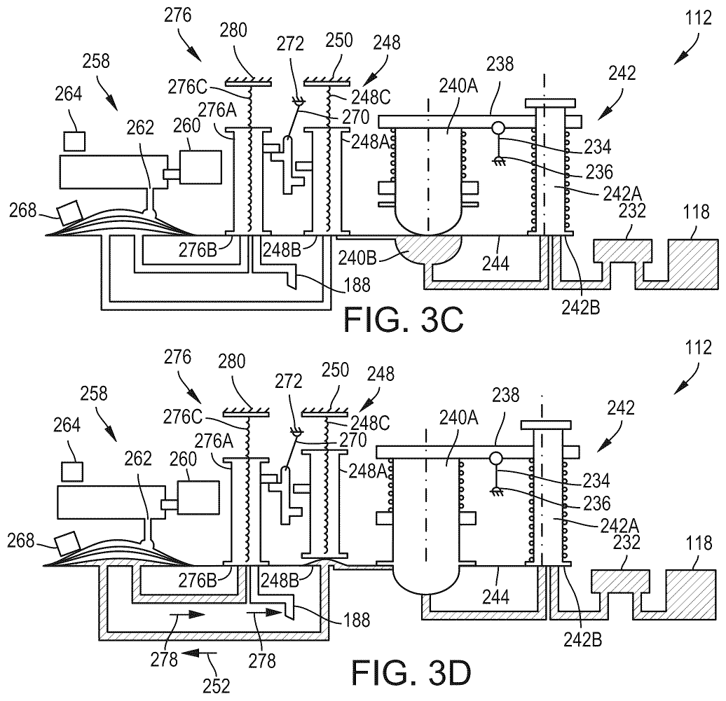

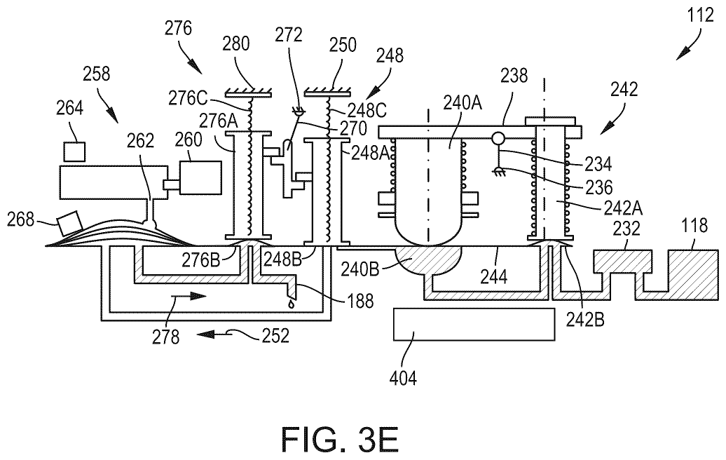

Referring now to FIGS. 2-3E, an actuation assembly 112 or arrangement which may be included in a dispensing assembly 100 (see, e.g., FIG. 1) is shown. The actuation assembly 112 depicted is an exemplary actuation assembly 112 and dispensing assemblies 100 may include any of a variety of actuation assemblies 112. Where a dispensing assembly 100 delivers multiple agents to one or more infusion devices 186, the reusable housing assembly 106 may include multiple of the actuation assembly 112 shown in FIGS. 2-3E each in communication with a separate agent reservoir 118.

In the example actuation assembly 112, an occluder assembly 232 (see, e.g. FIG. 3A) may isolate a filled reservoir 118 from the actuation assembly 112. Opening of the occluder assembly 232 may allow fluid to flow into the remainder of the actuation assembly 112. In order to effectuate the delivery of fluid within the reservoir 118 to the user, a controller 108 (see, e.g., FIG. 1) included within a dispensing assembly 100 may command energizing of a shape memory actuator 234, which may be anchored on one end using a shape memory actuator anchor 236. An opposing end of the shape memory actuator 234 may be coupled to a common connector 238 attached to a pump plunger 240A and reservoir valve assembly 242. Energizing of the shape memory actuator 234 may result in the activation of a pump 240 and the reservoir valve assembly 242. The reservoir valve assembly 242 may include a reservoir valve actuator 242A and a reservoir valve 242B. Activation of the reservoir valve assembly 242 may result in the downward displacement of the reservoir valve actuator 242A and the closing of the reservoir valve 242B, resulting in the effective isolation of the reservoir 118 from the actuation assembly 112. A membrane 244 may be included between a pump plunger 240A and a pump chamber 240B of the pump 240. The reservoir valve actuator 242A may press the membrane 244 against a valve seat of the reservoir valve 242B in order to close the reservoir valve assembly 242. Pump 240 and reservoir valve assembly 242 may be arranged and connected by the connector 238 whereby reservoir valve assembly 242 may close prior to the pump 240 pumping fluid. The activation of the pump 240 may result in the pump plunger 240A being displaced in a downward fashion into the pump chamber 240B leading to a displacement of the fluid (in the direction of arrow 246). The pump chamber 240B may be shaped to be substantially the same as the end of the pump plunger 240A in order to substantially empty the pump chamber 240B with each stroke of the pump 240.

A volume sensor valve assembly 248 may include a volume sensor valve actuator 248A and a volume sensor valve 248B. Referring also to FIG. 3B, the volume sensor valve actuator 248A may be maintained in a closed position via a volume valve spring assembly 248C (e.g. acting against a spring anchor 250) that provides mechanical force to move the volume sensor valve actuator 248A against the volume sensor valve 248B to seal volume sensor valve 248B. The volume sensor valve actuator 248A may press a membrane 244 included in the reservoir assembly 102 against a valve seat of the volume sensor valve 248B in order to close the volume sensor valve 248B. When the pump 240 is activated, however, if the displaced fluid is of sufficient pressure to overcome the mechanical sealing force of the volume sensor valve assembly 248, displacement of the fluid may occur in the direction of arrow 252. This may result in the filling of a volume sensor chamber 256 included within a volume sensor assembly 258 (shown in FIG. 3D). Through the use of a speaker assembly 260, port assembly 262, reference microphone 264, spring diaphragm 266, and variable volume microphone 268, the volume sensor assembly 258 may determine the volume of fluid within the volume sensor chamber 256. Operation of such a volume sensor assembly 258 may be as discussed in, for example, U.S. Pat. No. 8,491,570 issued Jul. 23, 2013 and entitled Infusion Pump Assembly (Attorney Docket No. G75) which is incorporated herein by reference in its entirety above. Other suitable dispensed volume sensors may be used in other embodiments.

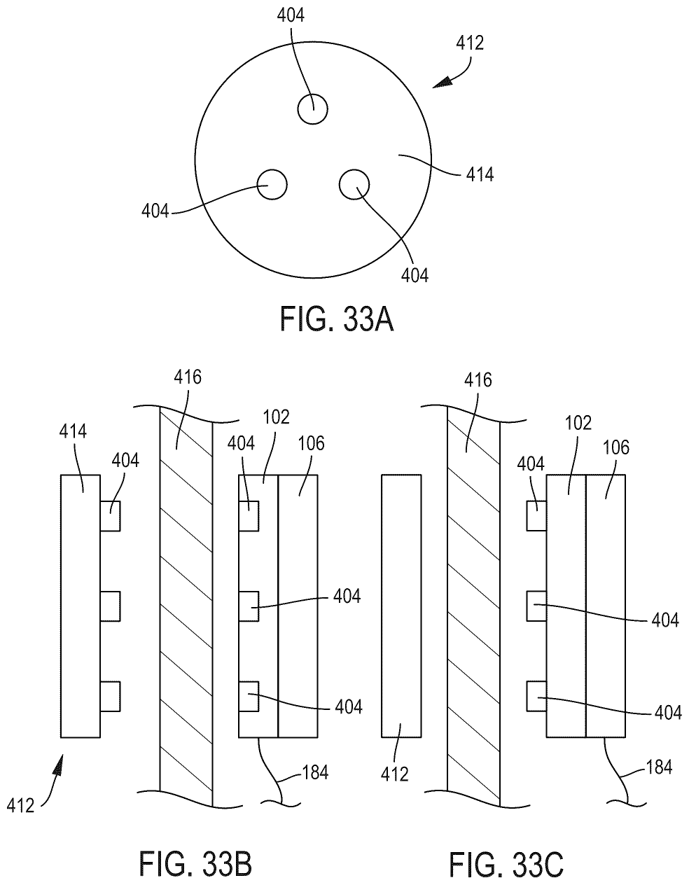

Referring also to FIG. 3D, a shape memory actuator 270 may be anchored (on a first end) to a shape memory actuator anchor 272. Additionally, the other end of the shape memory actuator 270 may be used to provide mechanical energy to a valve actuator 276A, which may activate a measurement valve assembly 276. Once the volume of fluid included within the volume sensor chamber 256 is calculated, the shape memory actuator 270 may be energized, resulting in the activation of measurement valve assembly 276. The measurement valve assembly 270 may include a measurement valve actuator 276A and a measurement valve 276B. Once activated to lift the measurement valve actuator 276A from the measurement valve 276B, due to the mechanical energy asserted on the fluid within volume sensor chamber 256 by the spring diaphragm 266, the fluid within the volume sensor chamber 256 may be displaced (in the direction of arrow 278) through a cannula 188 (e.g. of an infusion device 186) and into the patient 104. The measurement valve actuator 276A may then, by de-energizing the shape memory actuator 270 and by action of the measurement valve spring assembly 276C (e.g. acting against spring anchor 280) press a membrane included in the reservoir assembly 102 against a valve seat in order to close the measurement valve 276B. In some embodiments, the membrane interfaces 244 included over the reservoir valve 242B, pump chamber 240B, volume sensor valve 248B, and the measurement valve 276B may be formed in a single piece of material having regions overlying each of these components. Though the example embodiment is depicted with a cannula 188, any suitable access member may be used. In certain implementations the cannula 188 may be replaced by one or more microneedle or any array of microneedles. Additionally, as shown, the reservoir portion 102 may include one or more securement body which is depicted in FIG. 3E as a magnet 404. Securement bodies are further described, for example, in relation to FIGS. 33A-33C.

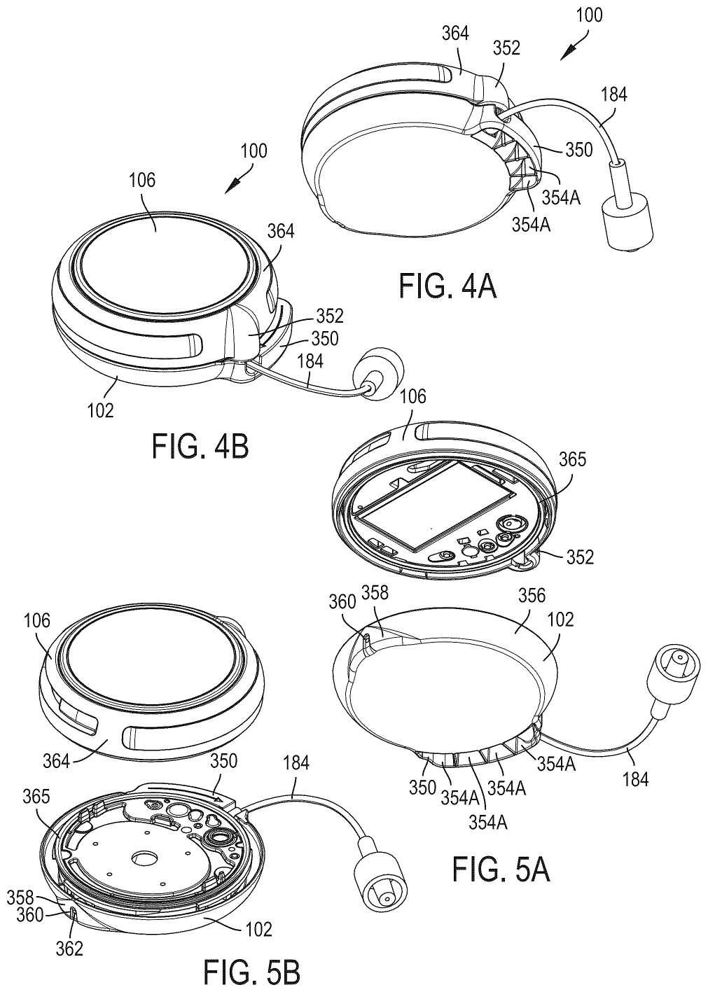

Referring now to FIGS. 4A-5B, an example embodiment of a dispensing assembly 100 is depicted. The dispensing assembly 100 is shown with the reservoir assembly 102 and a reusable housing assembly 106 coupled together in FIGS. 4A-B and exploded apart in FIGS. 5A-B. As shown, the dispensing assembly 100 may have a round footprint and be generally puck like in appearance. The reusable housing assembly 106 may be round and include a nub 352 which may project from the periphery of the exterior housing 364 of the reusable housing assembly 106. An environmental seal 365 may be provided such that interior portions of the dispensing assembly 100 are isolated from the surrounding environment when the reservoir assembly 102 and reusable housing assembly 106 are coupled to one another.

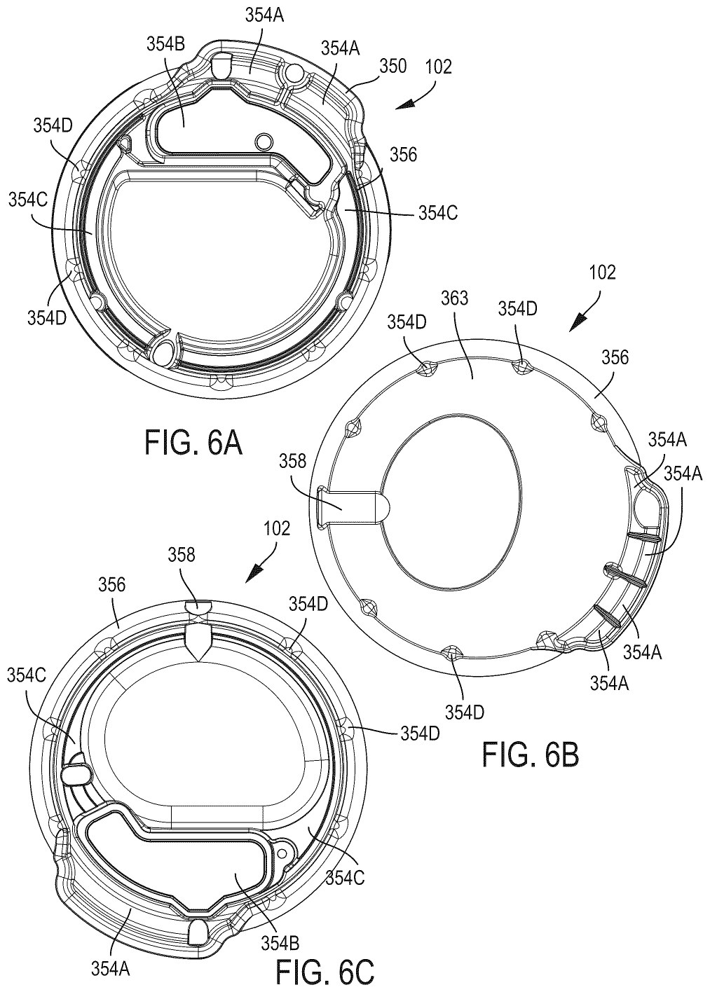

Referring now additionally to FIG. 6A-6C, which depict bottom plan views of a variety of exemplary reservoir assemblies 102, a reservoir assembly 102 be round and include a tab 350 which extends from a sidewall 356 of the reservoir assembly 102. At least one node region 358 may also be included and may be raised with respect to the adjacent portions of the sidewall 356. In some examples, node regions 358 may also extend onto other exterior faces of the reservoir assembly 102. A node region 358 extends onto a bottom face of the reservoir assembly 102 shown in FIG. 6B. Node regions 358 may be absent in other embodiments (see, e.g., FIG. 6A). Where included, a node region 358 may include a filling port 360 which may be used to load agent into a reservoir 118 of the reservoir assembly 102 (see, e.g., FIG. 5B). The filling port 360 may be sealed, for example, via a septum 362 in certain embodiments. In certain embodiments, the filling port 360 may be provided in an interior portion of the reservoir assembly 102 and the exterior face may be defined in a cover shell 363 which is coupled over the rest of the reservoir assembly 102 (which may be referred to as a cassette herein). An example embodiment including a shell 363 is depicted in FIG. 6B. In such examples, a node region 358 may be present, however, a filling port 360 may not be visible.

One exterior face (e.g. bottom face) of the reservoir assembly 102 may include at least one recess 354A-D. One or more recess 354A may for example be included on a face (e.g. underside) of the tab 350 as shown in FIG. 4A and FIG. 5A for instance. Such recesses 354A may, for instance, facilitate keeping wall thicknesses in the material forming the tab 350 substantially uniform. In the example shown, a number recesses 354A in the form of pockets are included in the face of the tab 350. In certain examples, there may also be a recess 354B in the form of a well surrounded by walls which may be disposed adjacent the tab 350 (see, e.g., FIG. 6A and FIG. 6C). In various examples, one of more recesses 354C may be provided in an exterior face adjacent the sidewall 356 of the reservoir assembly 102. In some examples, recesses 354D may be disposed at the transition between the exterior face and the sidewall 356 (see, e.g., FIGS. 6A-6C). The recesses 354A-D may be of uniform depth or one or more of the recesses 354A-D in a reservoir assembly 102 may be of variable depth. In some examples the recesses 354D may instead by raised bumps.

When coupled together, the reservoir assembly 102 and the reusable housing assembly 106 may be placed against one another with the nub 352 at one side of the tab 350. The reusable housing assembly 106 may be rotated such that the nub 352 is swung to the other side of the tab 350 to couple the reservoir assembly 102 and reusable housing assembly together (e.g. via a bayonet type coupling). This coupling action may also establish a seal via the environmental seal 365 of the reusable housing assembly 106. A compressible member for forming an environmental seal may additionally or alternatively be included on the reservoir assembly 102 as shown in FIG. 5B.

In various embodiments, a dispensing assembly 100 of a drug delivery system 10 may typically be portable and may accompany a patient 104 as the patient 104 goes about their quotidian activities. The dispensing assembly 100 may, for instance, be placed in a pocket of a garment worn by a patient 104. Alternatively, a dispensing assembly 100 may be adhered to a patient 104 directly via an adhesive pad 300 (e.g. borne by the reservoir assembly 102). In certain examples, a dispensing assembly 100 may couple to an intermediate component which may be adhered to a patient 104 via an adhesive pad 300. The intermediate component and coupled adhesive pad 300 may be referred to herein as a retention assembly. In such examples, the exterior surfaces of the reservoir assembly 102 may be adhesive free.

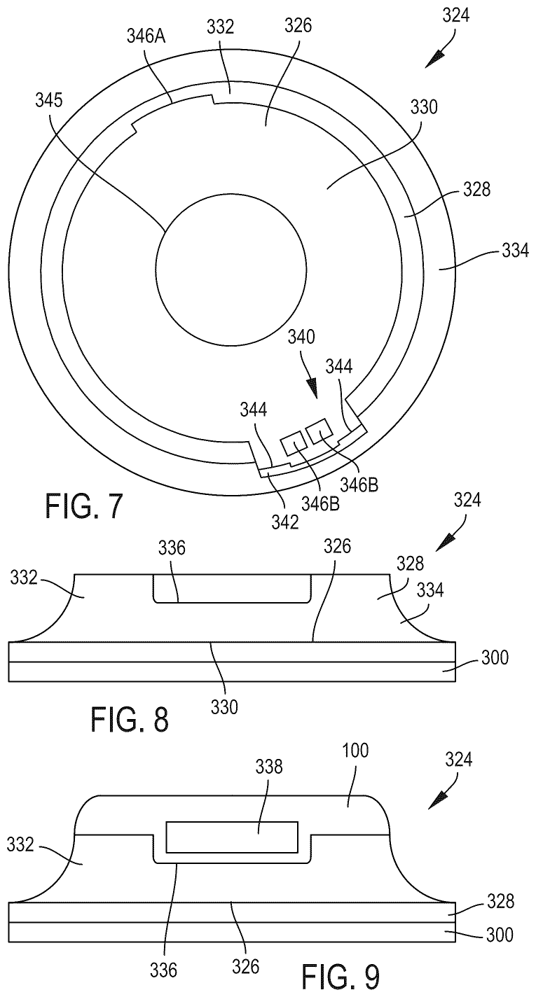

Referring now also to FIGS. 7-9, the intermediate component may be a socket 324 into which at least a portion of the dispensing assembly 100 may be seated and retained within. A socket 324 may include a rigid portion 326 and optionally a compliant portion 328. The rigid portion 326 may retain the dispensing assembly 100 and may be constructed of an injection molded plastic. The compliant portion 328 may be adhered to the patient 104 and have a footprint which is larger than that of the rigid portion 326. This may allow for portions of the socket 324 to bend and conform to the patient 104 when the socket 324 is adhered to the patient 104 and as the patient 104 moves about. The compliant portion 328 may, for example, be constructed of a silicone material. The compliant portion 328 may be overmolded onto the rigid portion 326 of the socket 324 in certain examples, though may be coupled to the rigid portion 326 in any other suitable manner. The rigid portion 326 may be adhered to the patient 104 in embodiments where a complaint portion 328 is absent.

Certain exemplary sockets 324 may include a base plate 330 which may be part of the rigid portion 326. Base plates 330 may optionally include a central aperture 345 through which underlying skin may be exposed and accessible. A wall 332 may project from the base plate 330 along at least one segment of the periphery of the base plate 330. Thus, the wall 332 may define part of a receptacle of the socket 324 into which the dispensing assembly 100 may be received. The wall 332 may be shaped to assist in lowering potential for the socket 324 to snag. For example, the exterior surface 334 of the wall 332 may be ramped such that the wall 332 increases in width as proximity to the base plate 330 increases. Though a curved exterior surface 334 is shown in the example, the exterior surface 334 may be at a constant slope in alternative embodiments. Thus the wall 332 may have a conic frustum type exterior surface 334. The wall 332 may be formed in the rigid portion 326 or formed as part of the complaint portion 328. In still other embodiments, the wall 332 may be partially formed of the rigid portion 326 and partially formed of the complaint portion 328. The interior portion of the wall 332 may, for instance, be formed from the rigid portion 326 and the exterior portion of the wall 332 may be formed of the complaint portion 328 in some examples.

As shown, the wall 332 may include one or more notches 336. A single notch 336 is shown in the example embodiment. The notch 336 may provide clearance for or line of sight to one or more user interface component of the dispensing assembly 100. In the example shown, the notch 336 provides clearance for a button 338 of the reusable housing assembly 106. A notch 336 may also be provided to create an opening for a speaker or illuminator included in the reusable housing assembly 106. In alternative embodiments, the notch 336 may be replaced by a fenestration in the wall 332 through which a button 338 or the like on the dispensing assembly 100 may be accessed. A notch 336 or fenestration may also be included to provide access to a septum 362 in a reservoir assembly 102.

As shown best in FIG. 7, the wall 332 may also include one or more breaks. That is, the periphery of the base plate 330 may include one or more wall free region 340. Wall free regions 340 may be included in place of notches 336 in certain examples. Additionally, a wall free region 340 may be included to accommodate or accept the tab 350 (see, e.g., FIG. 4B) of a reservoir assembly 102.

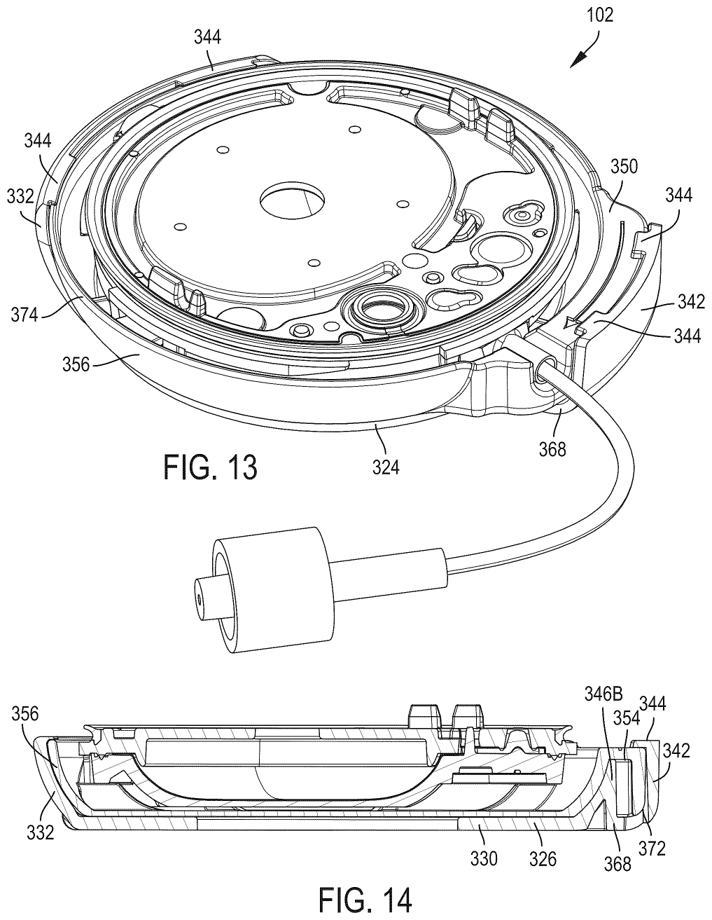

The socket 324 may include one or more clip 342. In the example, only a single clip 342 is depicted, however, any suitable number of clips 342 may be used in various embodiments. For example, symmetrically disposed clips 342 or clips disposed at regular angular intervals (e.g. 180°, 120°, 90°, etc.) may be included. A clip 342 may be formed as part of the rigid portion 326 of the socket 324. As a dispensing assembly 100 is seated into the socket 324, any clip(s) 342 may capture a surface (see, e.g., FIG. 13) of the dispensing assembly 100 such that the dispensing assembly 100 is retained in place within the socket 324. Each clip 342 may include at least one ledge 344 which may overhang a surface of the dispensing assembly 100 when the dispensing assembly 100 is in a retained state within the socket 324. In the example embodiment, the clip 342 is positioned so as to capture a portion of the tab 350 of the reservoir assembly 102. As the dispensing assembly 100 is advanced into the socket 324, the clip 342 may deflect and restore once the dispensing assembly 100 has been beyond a certain distance. A snapping noise or click may be produced as the clip 342 restores from its deflected state to provide an audible feedback indicating that the dispensing assembly 100 is retained in place within the socket 324. The clip 342 may be deflected by a user to disengage the clip 342 from the dispensing assembly 100 in order to remove the dispensing assembly 100 from the socket 324 as desired. This may allow a patient 104 to detach the dispensing assembly 100 from their body when desired. Thus, the dispensing assembly 100 may be removed to allow for a patient 104 to perform various activities such as swimming or showering. In embodiments where a central aperture 345 is included, the dispensing assembly 100 may also be removed to allow the user to access skin covered by the socket (e.g. to itch, scratch, allow open access to ambient air, etc.). Use of a socket 324 may also provide other benefits. Instead of directly adhering a reservoir assembly 102 to a patient 104, use of a socket 324 may allow the adhesive pad 300 to be replaced or moved without discarding the reservoir assembly 102 (which may contain expensive unutilized agent). A new socket 324 may be applied to the patient 104 and the dispensing assembly 100 may be installed in the new socket 324. Thus, a partially depleted reservoir assembly 102 may continue to be drawn from by the dispensing assembly 100 until it is fully depleted. This may be particularly desirable depending on the agent as some reservoir assemblies 102 may be loaded with agent sufficient to perform therapy for several days.

A socket 324 may include one or more alignment aids 346A, B which may assist in seating a dispensing assembly 100 into a socket 324. In some embodiments, the alignment aids 346A, B may be disposed in a poka-yoke arrangement. That is, the arrangement of the alignment aids 346A, B may inhibit or block installation of the dispensing assembly 100 in a socket 324 in orientations which do not correspond with a prescribed orientation. The alignment aids 346A, B may be formed as regions with features that are complimentary but substantially opposite to those of the dispensing assembly 100. For example, the alignment aids 346A, B may be raised or recessed regions on the socket 324 which may cooperate respectively with recessed or raised features on portions of the dispensing assembly 100. The alignment aids 346A, B may be formed in the rigid portion 326 or compliant portion 328 of the socket 324. In some embodiments, alignment aids 346A, B may be present in each of these portions.

As mentioned above in respect to FIGS. 4A-5B, in certain examples, the sidewall 356 of the reservoir assembly 102 may include at least one node region 358 which is proud of the adjacent surfaces of the exterior of the reservoir assembly 102. As shown, the socket 324 may include an alignment aid 346A which may accept the node region 358 when a dispensing assembly 100 is installed in the socket 324. The alignment aid 346A may be formed as a receptacle in the wall 332 of the socket 324. The alignment aid 346A may accept the node region 358 as the dispensing assembly 100 is installed in the socket 324. In certain embodiments, the wall 332 of the socket 324 may present an interference that inhibits installation or seating of the dispensing assembly 100 in the socket 324 in the event that the node region 358 is misaligned with the receptacle formed by the alignment aid 346A.

The reservoir assembly 102 may also include one or more recess 354A-D (see, e.g. FIGS. 4A-5B and FIGS. 6A-6C) on a face of the reservoir assembly 102 which may be adjacent the base plate 330 when the dispensing assembly 100 is retained in a socket 324. The socket 324 may include at least one alignment aid 346B which may extend into a complimentarily shaped recess 354A-D in the reservoir assembly 102. Preferably, the alignment aid(s) 346B may be formed such that they are incapable of fitting into other recesses 354A-D (in the event they are present) formed in the face of the reservoir assembly 102. Thus, the alignment aid(s) 346B may block seating of the dispensing assembly 100 within a socket 324 in the event that the dispensing assembly 100 is not in a prescribed orientation. In some embodiments, example alignment aid(s) 346B may be tapered such that their cross-sectional area decreases as distance from the base plate 330 increases. Such alignment aid(s) 346B may act as guides which may help to assist in bringing a dispensing assembly 100 into a proper orientation as the dispensing assembly 100 is placed into the socket 324. In the example shown, the alignment aids 346B depicted are positioned to extend into pockets defined on a face of the tab 350 (see, e.g., FIGS. 4A-5B) of the reservoir assembly 102.

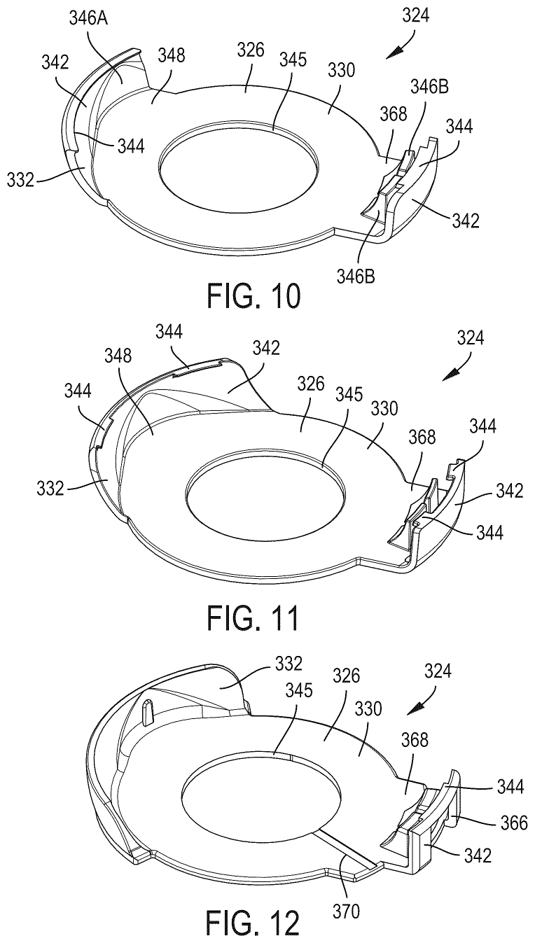

Referring now to FIGS. 10-12, a number of example sockets 324 are depicted. As shown, the sockets 324 include rigid portions 326 and are devoid of compliant portions 328. In alternative examples, any of the complaint portions 328 shown or described herein may be paired with the sockets 324 shown in FIGS. 10-12. As shown, each of the rigid portions 326 includes a base plate 330. The base plates 330 each include a central aperture 345. A wall 332 is shown extending from each of the base plates 330.

In the example embodiments, the wall 332 is depicted extending from an outcropped region 348 on the base plate 330. Including an outcropped region 348 on the base plate 330 may facilitate making the footprint of the base plate 330 relatively small. This may decrease the amount of material consumed to form the socket 324 and may also limit the amount of non-breathable material in close proximity to the patient 104 as the socket 324 is worn. As shown, the wall 332 may be present along the minority of the periphery of the base plate 330. The wall 332 may for example extend around a 120° or smaller section of the base plate 330. As mentioned in relation to FIGS. 7-9, the wall 332 may include an alignment aid 346A. The alignment aid 346A is shown as a recess which may accommodate the node region 358 of the reservoir assembly 102 when the reservoir assembly 102 is seated in the socket 324. The wall 332 may also include one or more ledges 344 and thus may double as a clip 342. The ledges 344 may be provided in discrete segments positioned along a portion of the wall 332 opposite the base plate 330 (see, e.g., FIG. 11) or a lip running along substantially the entirety of this portion of the wall 332 may provide the ledge 344 (see, e.g., FIG. 10).

The rigid portion 326 may also include an appendage 368 which may extend outward from the periphery of the base plate 330. A clip 342 may be defined at an edge of the appendage 368 most distal to the base plate 330. Thus, the clip 342 may be cantilevered from the base plate 330. This may facilitate user deflection of the clip 342 and potentially a portion of the appendages 368 when it is desired to release a dispensing assembly 100 from the socket 324. The clip 342 may include a window 366 which may be included to facilitate molding of the ledges 344 on the clip 342. The window 366 may also assisting in making it easier for a user to deflect the clip 342 in order to disengage the clip 342 from an installed dispensing assembly 100. In certain examples, multiple appendages 368 with clips 342 may be included. Similar windows may be included on the wall 332 to facilitate molding and release of the clip 342 provided by the wall 332.

Alignment aids 346B are also disposed on the appendage 368 in the examples shown. In the example embodiments, the alignment aids 346B are depicted as teeth like members which taper thinner as they increase in height. This may assist in guiding a dispensing assembly 100 into a proper orientation on the socket 324 as the dispensing assembly 100 is coupled to the socket 324.

Depending on the embodiment, the locations of the clips 342 on the socket 324 may be substantially symmetric about a midplane which extends through the center of the appendage 368 (see, e.g., FIGS. 11-12). In alternative embodiments (see, e.g., FIG. 10), the socket 324 may be asymmetric. It may be preferred that clips 342 be disposed substantially symmetrically about the socket 324 such that force needed to dislodge a dispensing assembly 100 is substantially equivalent when applied at corresponding locations on opposing sides of the midplane.

In certain examples, the base plate 330 may define one or more channels 370 which extend from a periphery of the base plate 330 to the central aperture 345. A channel 370 is depicted recessed into a face of the base plate 330 most distal to the patient 104, however, channels 370 may additionally or alternatively be included on the opposing face of the base plate 330. Any desired number of channels 370 may be included and may be disposed in regular angular intervals on a face of the base plate 330 in certain examples. The channels 370 may be included to provide flow pathways for air to and from the central aperture 345. This may help to increase breathability and comfort when a socket 324 is adhered to a patient 104. One of the channels 370 may also serve as an indicator which assists in providing a visual cue to the user that communicates the proper installation orientation for a dispensing assembly 102. Such a channel 370 may be visually different than other channels 370 on the same face of the base plate 330. Alternatively, the face of the base plate 330 which is visible when the socket 324 is adhered to a patient 104 may only have one channel 370 as shown and that channel 370 may act as the indicator. The indicator channel 370 may resemble, for example, tubing 184 or a portion of a fluid pathway extending from the reservoir assembly 102.

Referring now to FIGS. 13-14, a perspective view and cross-sectional view of a reservoir assembly 102 coupled in a socket 324 are respectively depicted. The reusable housing assembly 106 is not depicted in FIGS. 13-14 to better illustrate how clips 342 of a socket 324 may couple a dispensing assembly 100 in place on the socket 324. The clip 342 on the appendage 368 of the rigid portion 326 may deflect as the reservoir assembly 102 is pressed into the socket 324. The underside of the tab 350 may include a rounded edge surface 372 which may facilitate deflection of the clip 342 as the reservoir assembly 102 is placed into the socket 324. The clip 342 may snap in place with the ledge(s) 344 of the clip 342 overhanging the tab 350 when the reservoir assembly 102 is fully seated in the socket 324. Additionally, the reservoir assembly 102 may include a rim 374 at its periphery. The ledges 344 on the wall 332 may similarly snap in place over the rim 374 when the reservoir assembly 102 is fully seated in the socket 324. The sidewall 356 of the reservoir assembly 102 may be contoured such that the footprint of the reservoir assembly 102 is largest at the rim 374. This may facilitate deflection of the wall 332 as the reservoir assembly 102 is seated into the socket 324. There may be a small gap between the reservoir assembly 102 and the reusable housing assembly 106 to accommodate the ledges 344 when the reservoir assembly 102 and reusable housing assembly 106 are coupled together. Alternatively, the rim 374 of the reservoir assembly 102 may include recesses for each of the ledges 344. The recesses may have a depth such that when clipped in the socket 324, the ledges are flush with (or at least not proud of) the remainder of the rim 374.

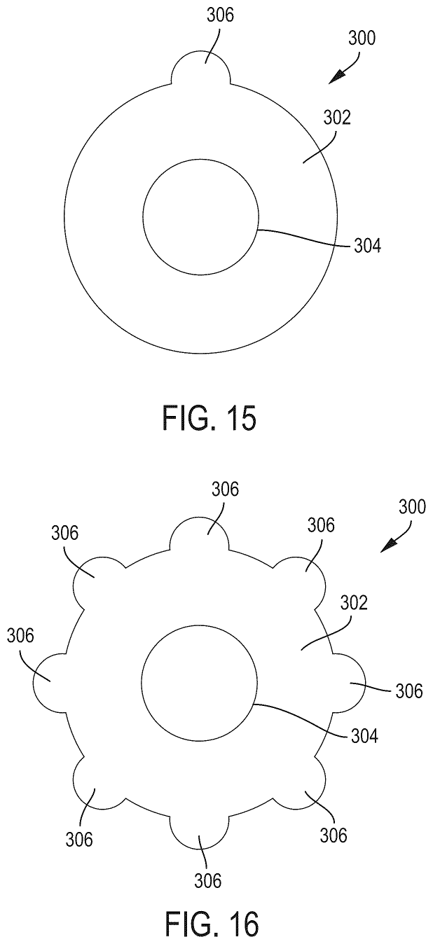

Referring now to FIGS. 15-17, an adhesive pad 300 may be coupled (e.g. adhered, sonically welded, heat staked, etc.) to an exterior surface (e.g. underside) of the socket 324. In some embodiments, the socket 324 may include energy concentrating ridges to facilitate sonic welding of the socket 324 to the adhesive pad 300. Such ridges may also be desirable where the adhesive pad 300 is coupled via a heat stake. Alternatively, the adhesive pad 300 may be adhesive bearing on both sides with a more aggressive adhesive being present on the socket 324 facing side of the adhesive pad 300. Thus, the adhesive pad 300 may be adhesively coupled to a socket 324. The adhesive pad 300 may be arranged to include one or more features which may assist in increasing comfort while the dispensing assembly 100 is worn over a period of several days. The example adhesive pads 300 may include a main region 302 which surrounds a central aperture 304. When the adhesive pad 300 is coupled to a socket 324, central aperture 304 of the adhesive pad 300 may be substantially aligned with any central aperture 345 in the socket 324 (see, e.g., FIG. 12). As a result, when the socket 324 is adhered to the patient 104, skin within the central aperture 304 may be directly exposed to air increasing comfort. Depending on the breathability of the material used to form the adhesive pad 300, no central aperture 304 may be included. Instead, the adhesive pad 300 may span across any central aperture 345 in the socket 324.

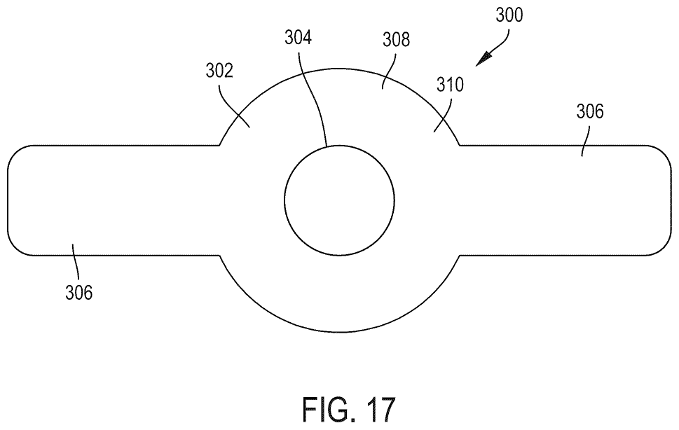

The adhesive pad 300 may include one or more peripheral protrusions 306. The peripheral protrusions 306 may be spaced (though need not in all embodiments) at regular angular intervals. The peripheral protrusions 306 may be any suitable shape or length, though in the example embodiment, are depicted as rounded nubs. The peripheral protrusions 306 may assist in facilitating removal of the adhesive pad 300 when a user is ready to remove the dispensing assembly 100 (e.g. to replace the reservoir assembly 102 after it is emptied). The peripheral protrusions 306 may extend beyond the footprint of the socket 324 such that they are easily accessible. In certain examples, the peripheral protrusions 306 may be nubs as shown in FIGS. 15-16. The peripheral protrusions 306 may alternatively be formed as a number of spoke members or arms as depicted in FIG. 17 for example. Such peripheral protrusions 306 may have lengths equal to at least 50% of the radius defining the outer periphery of the main region 302 (and up to 100%-250% or greater the length of that radius). The peripheral protrusions 306 may each be of equal length or may be of differing lengths. The lengths of the arm-like peripheral protrusions 306 make them more amenable to being adhered around contours of a patient's 104 body when a socket 324 is placed on the skin. This may assist in firmly anchoring the socket 324 to the patient 104 and inhibiting inadvertent dislodgement of the socket 324 from a patient 104.

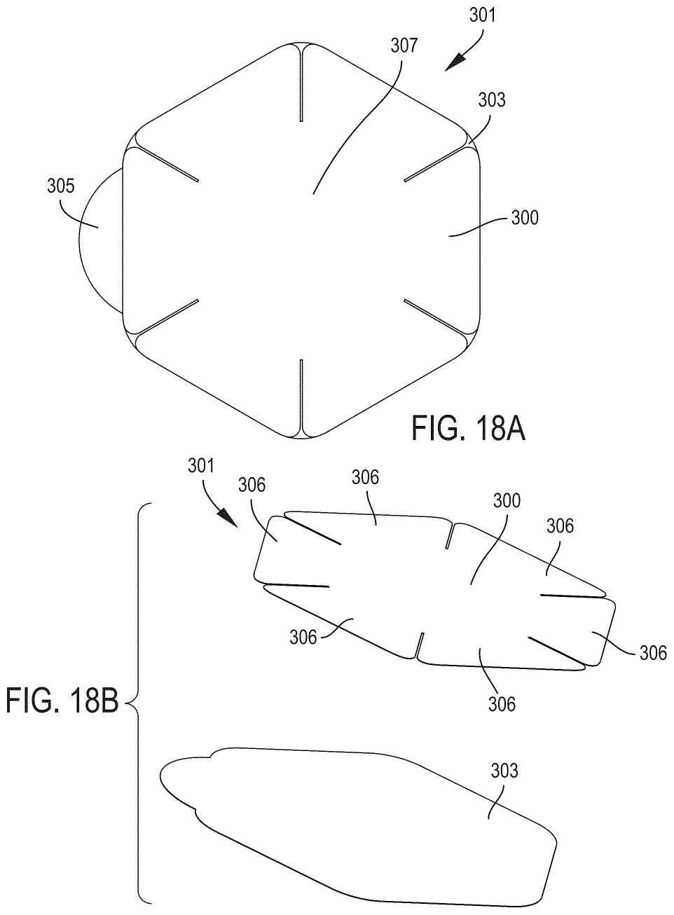

The adhesive pad 300 may be constructed of a variety of materials. In some embodiments, the adhesive pad 300 may be constructed of a woven fabric material. In other embodiments, the adhesive pad 300 may be constructed of a non-woven (e.g. spunbound or spunlaced material). The adhesive pad 300 may be constructed of synthetic fiber or natural fiber material. In other embodiments, a perforated polymer bandage type material may be used. The material may be adhesive bearing and the adhesive may be a skin compatible, medical grade adhesive. The adhesive pad 300 may be provided with the adhesive covered by a liner 303 or peelable backing which may protect the adhesive on the adhesive pad 300 prior to use (see, e.g., FIG. 18B). The liner 303 may be split into one or more pieces to facilitate facile removal.

Referring now to FIGS. 18A-18B, a top plan and exploded view of an exemplary adhesive assembly 301 are respectively depicted. As shown, the adhesive assembly 301 may include a release liner 303 and an adhesive pad 300. The release liner 303 may cover and protect the adhesive on the adhesive pad 300 until just prior to application of the adhesive pad 300 on a user. The release liner 303 may be constructed of any suitable material such as any of those used for band-aid peelable backings. The release liner 303 may include at least one grasping projection 305 which may facilitate removal of the release liner 303 from the adhesive pad 300. As shown, the example adhesive pad 300 includes a central region 307 which is uninterrupted and includes no central aperture 304. In other example a central aperture may be present. The adhesive pad 300 also includes a number of peripheral protrusions 306 which extend from the central region 307. In the example shown in FIGS. 18A-18B, the peripheral protrusions 306 are depicted as wings which increase in width as distance from the central region increases. In some examples, the adhesive pad 300 may be constructed as a single body (e.g. roughly hexagonal body) and the peripheral protrusions 306 may be formed in a kiss cut operation.

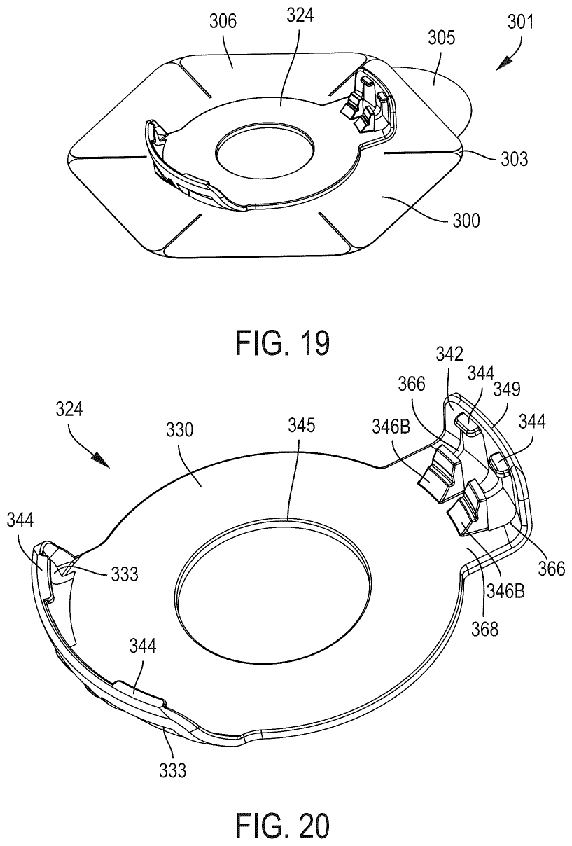

Referring now to FIGS. 19-20, another embodiment of a socket 324 is depicted. As shown in FIG. 19, the socket 324 may be attached to the central region 307 of the adhesive pad 300. The socket 324 includes a central aperture 345 in its base plate 330, but may be a continuous, uninterrupted piece of material in other examples. As shown best in FIG. 20, the wall 332 of the socket 324 may include fenestrations 333. The fenestrations 333 may facilitate molding of the ledges 344 on the wall 332 and may allow the wall 332 to more easily be flexed to engage/disengage a reservoir assembly 102. One of the fenestrations 333 may also provide an access to a septum 362 included in a reservoir assembly 102. In other examples, the fenestrations 333 may be positioned such that the wall obstructs access to the septum 362. The clip 342 on the appendage 368 may include a raised lip 349 which projects more distal the base plate 330 than the ledges 344 of the clip 342. This may provide an easier grasp point for deflecting of the clip 342 out of engagement with the tab 350 of the reservoir assembly 102. As shown, the clip 342 includes a set of windows 366 in line with the ledges 344 to facilitate molding thereof. The windows 366 may also increase the ease with which the clip 342 may be deflected to remove the reservoir assembly 102 from the socket 324.

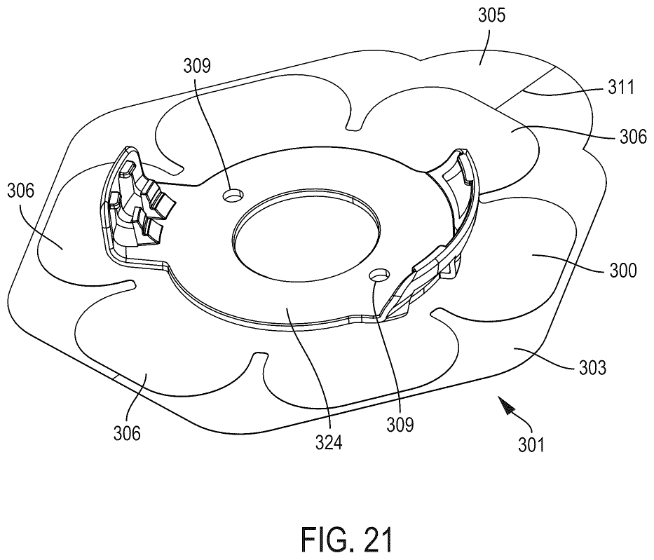

Referring now to FIG. 21, another example socket 324 coupled to an adhering assembly is depicted. As shown, the socket 324 and adhesive pad 300 may include pass-throughs 309. The pass-throughs 309 may facilitate alignment of the components during manufacture. The pass-throughs 309 may also provide additional paths for air to access the skin when the adhesive pad 300 is adhered to a patient. The peripheral protrusions 306 on the example adhesive pad in FIG. 21 are a set of rounded lobes or petal members which are positioned at regular angular intervals about the central region of the adhesive pad 300. A gap is present between each of the peripheral protrusions 306. Such peripheral protrusions 306 may make the adhesive pad 300 easier to disassociate from the release liner 306. They may further assist in preventing peripheral protrusions 306 and sticking to one another during handling. The release liner 303 is also shown with a slit 311 dividing the release liner 303 into individually removable sections. In the example, the slit 311 splits the release liner 303 in half and extends through the grasping projection 305. Slits 311 may be included in any release liner 303 shown or described herein and may split the release liner 303 into any desired number of individually removable parts.

Referring now to FIGS. 22-24, exemplary compliant portions 328 of sockets 324 are depicted. The compliant portions 328 are shown divorced from rigid portions 326 though could be coupled onto any rigid portions 326 shown and described herein. Where a compliant portion 328 is included, the adhesive pad 300 for a socket 324 may be coupled to the compliant portion 328. In some examples, no rigid portion 326 may be included and the socket 324 may be constructed solely of compliant material.

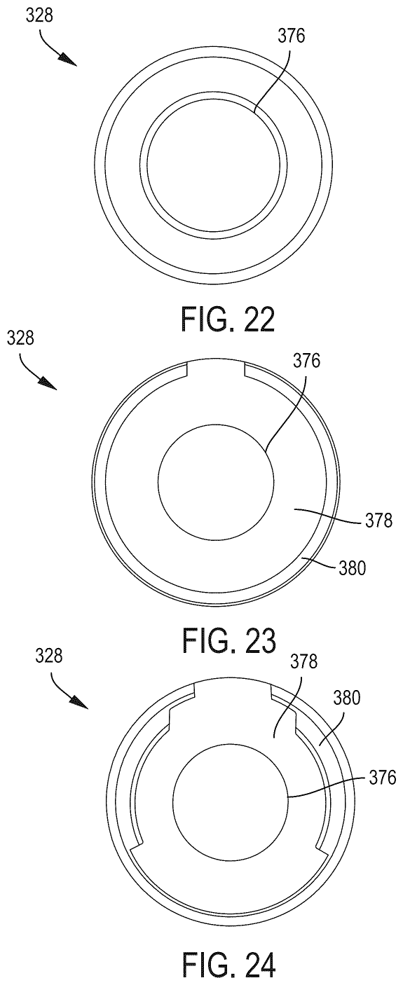

As shown in FIG. 22, a complaint portion 328 may include a central aperture 376. The compliant portion 328 may be coupled to the underside of a base plate 330. The central aperture 376 may match that of the base plate 330 such that an air path is established to the skin when the socket 324 is worn. In other examples (see, e.g., FIGS. 23-24), the complaint portion 328 may define a receptacle 378. The receptacle 378 may have walls arranged to match the shape of the footprint of the rigid portion 326. A rim 380 may at least partially surround the receptacle 378. The rim 380 may have a height which places the rim 380 substantially even with or proud of the base plate 330 when the compliant portion 328 and rigid portion 326 are coupled.

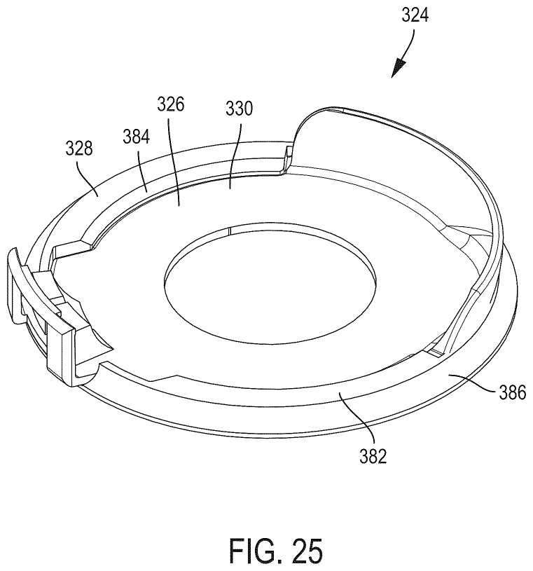

Referring now to FIG. 25, a perspective view of a socket 324 including a rigid portion 326 and compliant portion 328 is depicted. The complaint portion 328 may be overmolded onto the rigid portion 326. The complaint portion 328 may be molded so as to at least partially encase one side of the base plate 330 and may have a flange 382 which surrounds at least a portion of the base plate 330. The flange 382 may be at least even height with a side of the base plate 330 opposite that encased by the compliant portion 328 in the overmolding operation. In the example shown, the flange 382 is raised proud of the base plate 330. In some embodiments, an innermost section 384 of the flange 382 directly adjacent the edge of the base plate 330 may taper toward the base plate 330. Thus the innermost section 384 of the flange 382 may accept and cradle a portion of the sidewall 356 of a reservoir assembly 102 when the dispensing assembly 100 is retained in the socket 324. The outermost section 386 of the flange 382 may also taper or be rounded so as to limit potential snagging of the socket 324 on clothes or other objects.

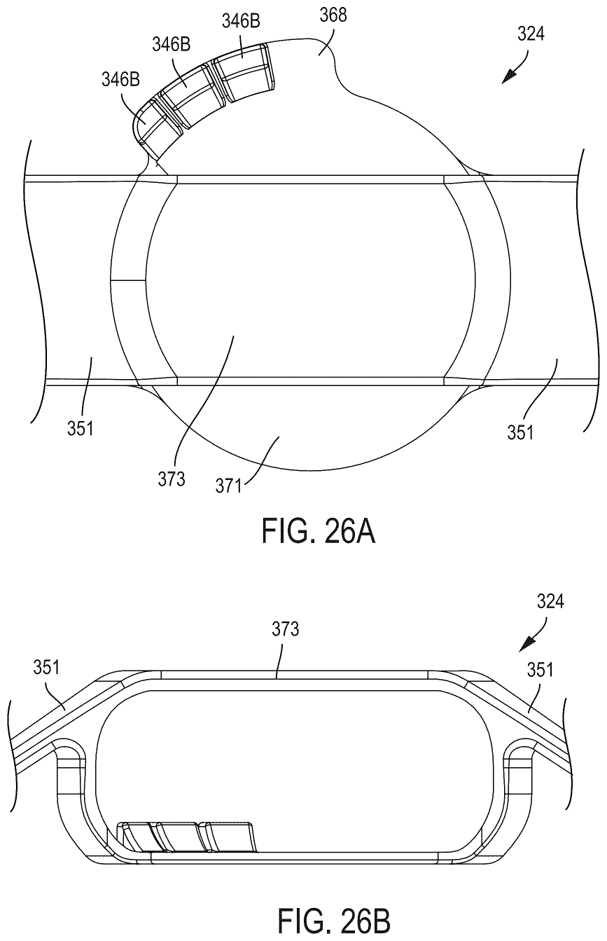

Referring now to FIGS. 26A-28B, a number of exemplary sockets 324 are depicted. The sockets 324 include no rigid portion and instead are formed entirely of a flexible material. The material may be elastomeric in certain examples. The sockets 324 may be free of clipping or latching features. Instead, the material may be slightly stretched when a dispensing assembly 100 is placed in the socket 324. Thus, the dispensing assembly 100 may be compressively held in place within the socket 324. In some examples, the socket 324 material may not be stretched when a dispensing assembly 100 is in place, however, may require stretching to remove the dispensing assembly 100. The dispensing assembly 100 would be retained in the socket 324 absent exertion of force to stretch the socket 324 material to allow for removal. The sockets 324 depicted in FIGS. 26A-28B also include no adhesive assembly 301 (though alternative embodiments may include an adhesive assembly 301). Instead, each of the sockets 324 includes a set of arms 351. The arms 351 may be disposed at regular angular intervals. In the examples depicted in FIGS. 26A-28B, each socket 324 includes a pair or arms 351 disposed opposite one another. The arms 351 are shown in truncated fashion in FIGS. 26A-28B. In some embodiments, the arms 351 may include openings at their terminal ends to accept part of a strap. In such examples, the sockets 324 may be worn as a watch type item. The strap may be elastomeric or adjustable so as to tightly hold the socket 324 in place on a user (e.g. arm/leg). The example sockets 324 may be particularly useful during athletic activities.

Referring primarily to FIGS. 26A-26B, the example socket includes a base 371. The base includes an appendage 368 with a set of alignment aids 346B. The alignment aids 346B may cooperate with recesses 354A of various reservoir assemblies 102 when a dispensing assembly 100 is retained by the socket 324. In the event that a reservoir assembly 102 is misaligned, the base 371 of the socket 324 may not sit flush against the surface of the reservoir assembly 102. This would provide a visual cue to a user that the dispensing assembly 100 has not been properly seated in the socket 324. Additionally, a tactile cue would be perceivable due to the unevenness of the base 371 against the reservoir assembly 102 should a user attempt to wear the socket 324 with the dispensing assembly 100 improperly seated.

The example socket 324 in FIGS. 26A-26B includes a band 373 of material. The dispensing assembly 100 may be advanced into the socket 324 such that the band of material surrounds the widest portion of the dispensing assembly 100. The dispensing assembly 100 may be compressively held in place by the elastomeric material forming the band 373. As shown, the arms 351 of the socket 324 extend from the band 373 in the example embodiment.

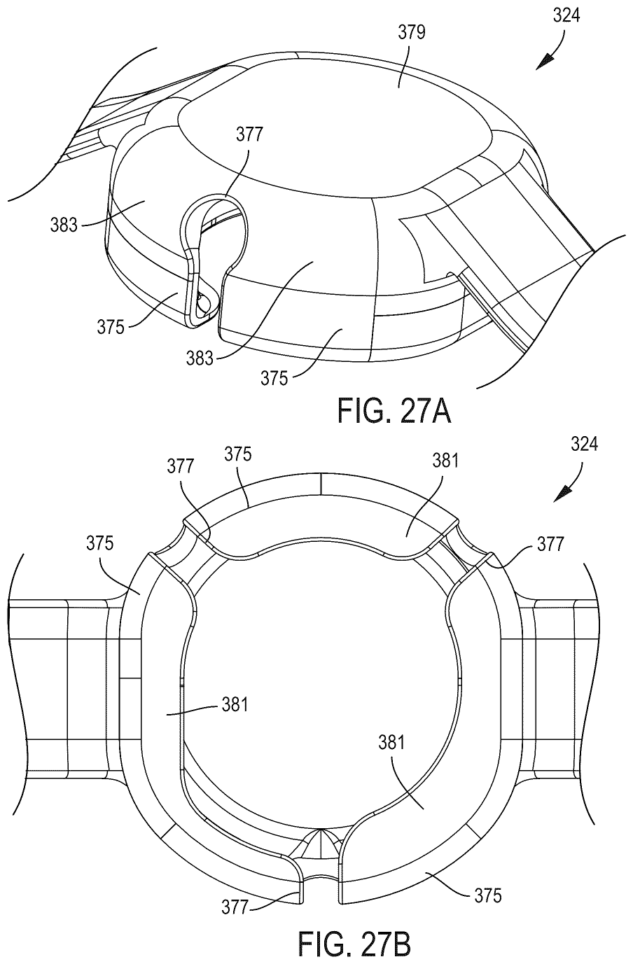

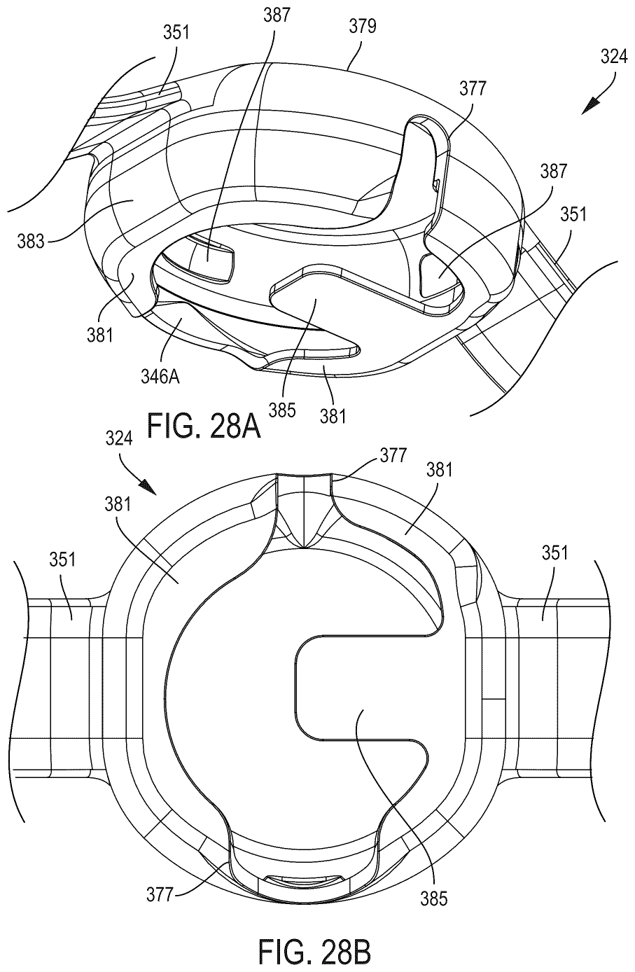

As shown in FIG. 27A-27B, an example elastomeric socket 324 is depicted. As shown, the socket 324 may be a sleeve for the dispensing assembly 100 and form a pocket within which the dispensing assembly 100 may be retained. The sleeve may include a set of wall segments 375 which extend from a main portion 379 of the sleeve. The wall segments 375 may be separated by notches 377 extending through the sleeve. Each of the wall segments 375 may be flexed/stretched independent of the others. Three wall segments 375 are included in the example shown, however, any suitable number may be included in alternative embodiments. The wall segments 375 include a terminal portion 381 which is substantially parallel to the main portion 379 and an intermediate portion 383 which spaces the terminal portion 381 from the main portion 379.

To install a dispensing assembly 100 into the socket 324, the wall segments 375 may be distorted to clear an approach for the dispensing assembly 100 into the pocket defined by the sleeve. The terminal portions 381 of the wall segments 375 may form graspable tabs which may help facilitate manual distortion of the sleeve. The dispensing assembly 100 may be inserted and the wall segments 375 may be allowed to restore to or toward a resting state. In some embodiments, the dispensing assembly 100 may be compressively retained within the socket 324 after the wall segments 375 restore toward their resting state. In other examples, the dispensing assembly 100 may not be compressively held by the socket 324, but the wall segments 375 may be sufficiently resilient to resist substantial stretching during strenuous exercise. As shown, the arms 351 extend from the main region 379. In some examples, the elastomeric material for the socket 324 may be overmolded to a rigid frame or skeleton to provide additional resiliency.

As shown, at least one of the notches 377 may be sized to accept a protruding portion of a dispensing assembly 100. For example, the notch 377 visible in FIG. 27A may accept the nub 352 of a reusable housing assembly 106 (see, e.g., FIG. 4B). The notch 377 may also provide an exit path for tubing 184 (where included) from the reservoir assembly 102. The notch 377 may also ensure that the dispensing assembly 100 may not be installed into the socket 324 without the reusable housing assembly 106 and reservoir assembly 100 properly coupled to one another. As described elsewhere herein, the nub 352 may be swung across the tab 350 of a reservoir assembly 102 during coupling. In the event the nub 350 has not be rotated to its fully coupled position, the nub 352 would be out of alignment with the notch 377. The dispensing assembly 100 would not seat appropriately within the socket 324 providing a cue to the user that adjustment is necessary. Notches 377 may also provide openings through which user interface components of a dispensing assembly 100 may be accessible. For example, buttons on the reusable housing assembly 106 may be accessed via the notches 377. A notch 377 may also be positioned in the vicinity of the output of a speaker of the reusable housing assembly 106 so as to help ensure that the socket 324 does not muffle sounds generated by the dispensing assembly 100.

Referring now primarily to FIGS. 28A-28B, another elastomeric socket 324 is depicted. As shown, in some examples, only two wall segments 375 may be included. At least one of the terminal regions 381 of the wall segments 375 may include a tongue 385 or other projection. The tongue 385 may assist in providing a grasp point for a user to distort the wall segments 375. Additionally, as best shown in FIGS. 28A-28B, elastomeric sockets 324 (such as any of those shown herein) may include alignment aids 346A. The alignment aid 346A may accept a node region 358 of the reservoir assembly 102. An elastomeric socket may also include recesses 387 on the interior surface which abuts the dispensing assembly 102. The recesses 387 may receive raised features of the reusable housing assembly 106. The recesses 387 may also act as alignment aids. In certain examples, at least one of the recesses 387 may be associated with a raised region or bump on the exterior face of the socket 324. The raised bump may indicate to a user where a user interface component on the reusable housing assembly 106 is located. As the socket 324 is formed of an elastomeric, flexible material, force may be applied to the bump to operate a button or the like on the dispensing assembly 100 retained within the socket 324. An aperture could alternatively be included for any such user interface components.

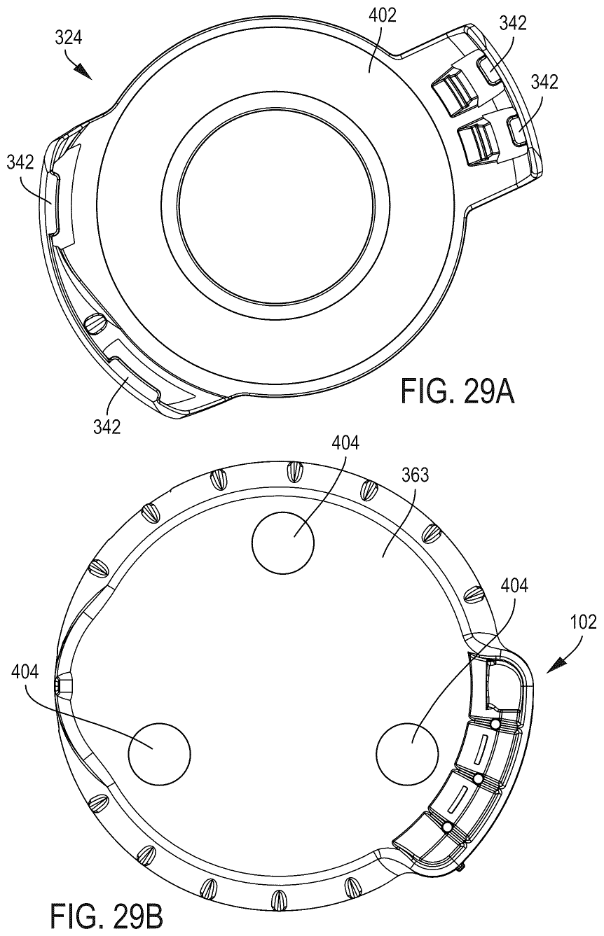

Referring now to FIGS. 29A-32, in certain embodiments, a dispensing assembly 100 may be attached to a patient via a magnetic coupling. For example, a retention assembly with an adhesive pad 300 and an intermediate component may magnetically attract or be attracted to part of the dispensing assembly 100. Any of the sockets 324 described herein may include one or more ferromagnetic region 402 or one or more magnet 404. Dispensing assemblies 100 having a shape memory alloy based actuation arrangements may be particularly well suited for retention assemblies incorporating magnets 404.

As shown in FIG. 29A, a ring of ferromagnetic material 402 is included as part of an example socket 324. Though a ring is depicted, a socket 324 may also include one or more individual regions of ferromagnetic material. The ferromagnetic material 402 may be adhered to the remainder of the socket 324 though may be attached or coupled to rest of the socket 324 in any other suitable manner. The reservoir assembly 102 of a dispensing assembly 100 may include at least one magnet 404 which may interact with the ferromagnetic material 402 in the socket 324 to retain the dispensing assembly 100 in place on the socket 324. A reservoir assembly 102 may include receptacles in the exterior face of the shell 363 into which the magnets 404 may be adhered. As a non-limiting example, a reservoir assembly 102 with three magnets 404 disposed at regular angular intervals is depicted in FIG. 29B. A greater or lesser number of magnets 404 in any suitable arrangement or pattern may be included in other embodiments. Though the example socket includes clips 342, in certain embodiments, the magnets 404 used may provide sufficient attraction that the clips 342 may be omitted. As described elsewhere herein, the socket 324 may be coupled to an adhesive pad 300. The magnetic retention arrangement may allow a user to attach and remove the dispensing assembly 100 form the socket 324 quickly without having to throw away or discard an adhesive pad 300. Additionally, the magnetic arrangement may assist in blindly locating the dispensing assembly in relation to the socket 324 may effectively guide the dispensing assembly 100 into place when brought into proximity of the socket 324.

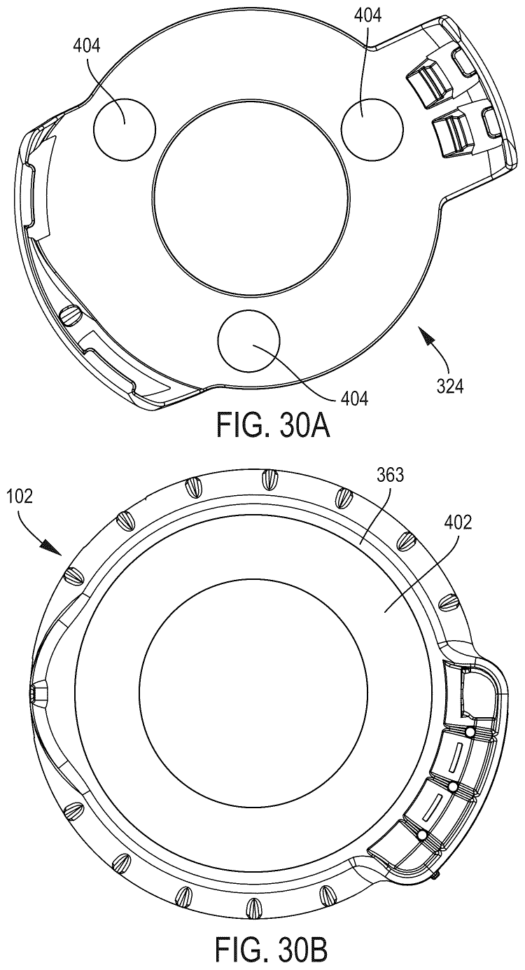

The ferromagnetic material on the socket 324 could be replaced by a permanently magnetized piece of material in alternative examples. For instance, an annularly shaped magnet 404 could be used instead. Additionally, and as shown in FIG. 30A, one or more individual permanent magnets 404 could be spaced about the socket 324. The number of magnets 404 on the socket 324 may, though need not necessarily, match the number of magnets 404 on the reservoir assembly 102. In some embodiments, the polarity of the magnets 404 on the socket 324 and the reservoir assembly 102 may be such that the reservoir assembly 102 is prevented from being attached to the socket 324 other than in a certain prescribed orientation or orientations.

In alternative examples, and as shown in FIG. 30B, the reservoir assembly 102 may include at least one ferromagnetic region 402. In such examples, the socket 324 may include one or more magnet 404. In the example, a ring of ferromagnetic material is included on the reservoir assembly 102. Any number of individual, discrete ferromagnetic regions 402 may also be used. The shell 363 may include recesses into which respective pieces of ferromagnetic material may be adhered or otherwise coupled during manufacture.

The magnets 404 may be any variety of magnet. In preferred examples, the magnets 404 used may be magnets formed from materials including elements in the lanthanide series. Rare earth magnets such as neodymium or samarium cobalt may be included. This may help to keep a retention assembly and reservoir assembly 102 compact while still firmly holding a dispensing assembly 100 in place.

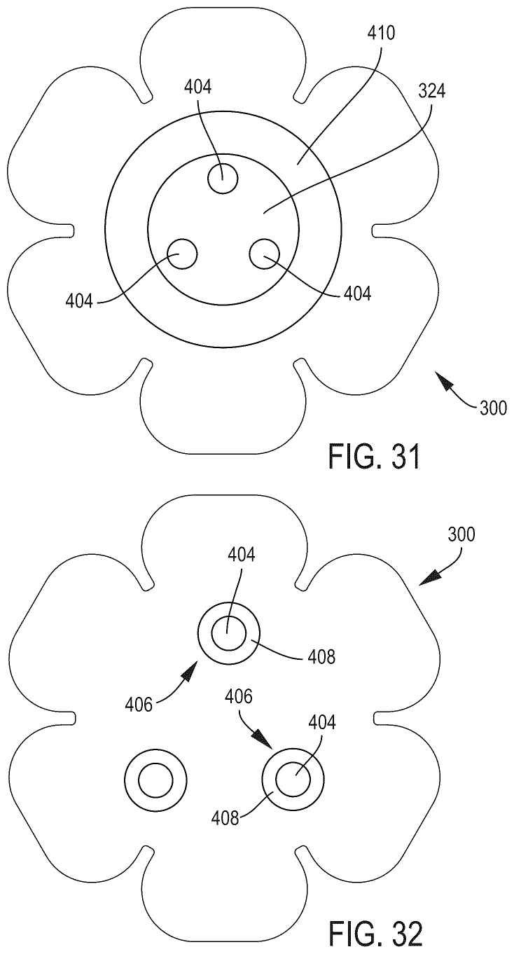

Referring now to FIG. 31, an embodiment of an adhesive pad 300 coupled to an example socket 324 is depicted. The socket 324 may include a rim 410 and be shaped as a dish or cradle which may substantially match the shape of the exterior of a reservoir assembly 102. No clips 342 are included. Instead, a number of magnets 404 are included in the socket 324. A reservoir assembly 102 such as that shown in FIG. 29B or FIG. 30B may be placed into the socket 324 and retained in place via magnetic attraction. In alternative examples, the socket 324 may include at least one ferromagnetic region 402 and may couple to a reservoir assembly 102 via magnets included in the reservoir assembly 102. Though shown as a round bowl, the socket 324 may be shaped to include a protruding region which snuggly accommodates a tab 350 of a reservoir assembly 102. A notch or other cutout may be present so that tubing 184 from the reservoir assembly 102 may be conveniently routed out of the socket 324 when the dispensing assembly 100 is coupled to the socket 324.

Referring now to FIG. 32, in some implementations, a socket 324 may not be used. Instead, the intermediate component of the retention assembly may be one or more magnetic pucks 406 which may be coupled to an adhesive pad 300. Each magnetic puck 406 may be include a magnet 404 coupled to a puck housing 408 which may be formed of a polymer material. The puck housing 408 may, in turn, be coupled to the adhesive pad 300. In some embodiments, the puck housing 408 may include a set of ribs (not shown) on a face thereof which may assist in sonically welding each puck 406 to an adhesive pad 300. The pucks 406 may be spaced about the adhesive pad 300 so as to match the locations of magnets 404 on a reservoir assembly 102 or align with one or more ferromagnetic regions 402 on the reservoir assembly 102. By including relatively small pucks 406 instead of a socket 324, the face of the adhesive pad 300 opposite the patient may be substantially uncovered. As the pucks 406 may create a short stand-off which helps to keep a path for air to flow under the dispensing assembly 100 when the dispensing assembly 100 is coupled to the retention assembly. This may help to increase patient comfort, especially when the retention assembly is being worn for prolonged periods. Though individual pucks are shown, in alternative embodiments, a ring shaped ferromagnetic region 402 or magnet 404 in a ring shaped housing may also be attached to an adhesive pad 300.

Referring now to FIG. 33A-33C, in some embodiments, no socket 324 or adhesive pad 300 may used. Reservoir assemblies 102 may be provided with a retainer 412. The retainer 412 may be a plate 414 to which one or more magnet 404 has been coupled as shown in FIG. 33A. Alternatively, the entire retainer 412 may be a magnetic body (see, e.g., FIG. 33C). A reservoir assembly 102 may be assembled to include a one or more magnets 404 or ferromagnetic regions 402. Alternatively, reservoir assemblies 102 may be provided with one or more ferromagnetic body or magnet 404 that may be adhered to the reservoir assembly 102 by a user (see, e.g., FIG. 33C). The magnetic or ferromagnetic components included in or adhered to the reservoir assembly 102 may be referred to herein as securement bodies. Magnetic attraction between the retainer 412 and the securement bodies may allow the dispensing assembly 100 to be coupled to an article of clothing (e.g. shirt, bra, shapewear, undergarment, strap or band around an appendage) worn by a patient. For example, the retainer 412 may be placed inside or outside of a garment 416 and the reservoir assembly 102 may be aligned with the retainer 412 on the opposing side of the garment 416. The garment 416 may be firmly captured between the reservoir assembly 102 and retainer 412 and keep the dispensing assembly 100 associated with the user as the user goes about their day.

The magnet(s) 404 used in the retainer 412 and/or securement bodies may be sufficient to firmly hold a dispensing assembly in place when a gap of at least 1 mm which is filled with garment material is disposed intermediate the retainer 412 and securement bodies. Any garment material such as leather, faux leather, padding, foam, latex, natural fiber, synthetic fiber, elastane or other elastomeric fibers, blends thereof, etc. may be present in the gap and the magnetic attraction between the retainer 412 and securement bodies may be sufficient to keep the dispensing assembly 100 in place. In various embodiments, neodymium or other rare earth magnets may be used.

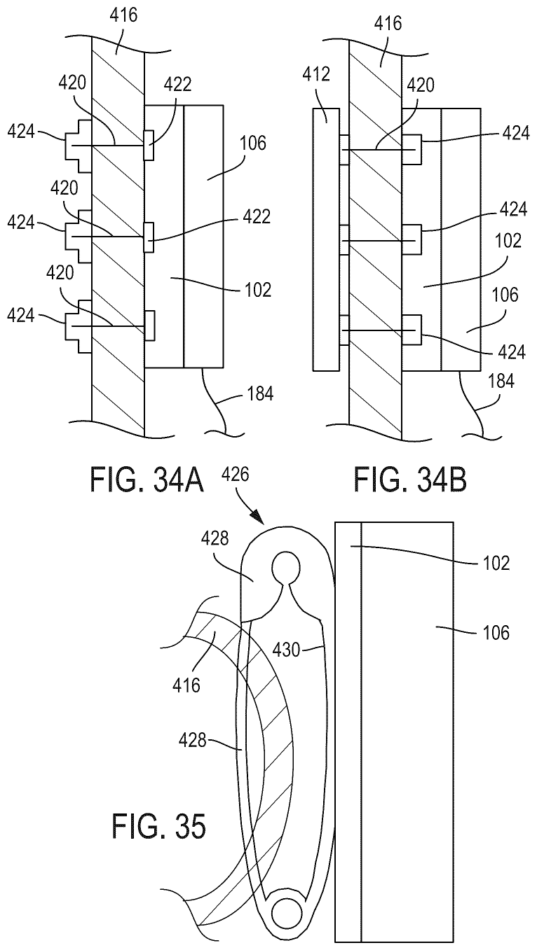

Referring now to FIG. 34A, in certain example embodiments, a reservoir assembly 102 may include one or more retention post 420. In the example, three retention posts 420 are depicted. The retention posts 420 may be coupled to the reservoir assembly 102 in any suitable fashion. In some examples, a portion of the reservoir assembly 102 may be overmolded onto the retention posts 420. In other embodiments, the retention posts 420 may be coupled to the reservoir assembly 102 via adhesive, may be pressed into snap fit or interference fit engagement with the reservoir assembly 102, placed into a depression with a raised surrounding rim which is swaged over to retain each retention post 420, etc. The retention posts 420 may include a base portion 422 which may be the portion of the retention post 420 coupled to the reservoir assembly 102. The retention posts 420 may include an end most distal to the reservoir assembly 102 which may be pointed. The retention posts 420 may extend through a garment 416. A respective clutch 424 may engage a region each retention post 420 most distal to the reservoir assembly 102 on a side of the garment 416 opposite the reservoir assembly 102. Any suitable retention post 420 and clutch 424 arrangement may be used. For example the retention posts 420 may be paired with a rubber clutch or butterfly clutch in certain embodiments. Retention posts 420 may alternatively be paired with a clutch 424 which locks in place on the retention post 420. Where a locking clutch is used, the clutch 424 may be a jewelry clutch, ball locking clutch, or flathead locking clutch.

In some examples, and as shown in FIG. 34B, a retainer 412 may be included. In such examples, at least one retention post 420 may extend from the retainer 412 which may be a plate like item. In the example depicted, the retainer 412 includes three retention posts 420. The retention posts 420 may be attached to the retainer 412 via overmolding, adhesive, press fit, snap fit, swaging, etc. The reservoir assembly 102 may have coupled thereto one or more clutch 424. The retention posts 420 may be pointed and extend through a garment 416. The portion of each retention post 420 protruding through a garment may be engaged with a clutch 424 on the reservoir assembly 102. Thus, the reservoir assembly 102 and any attached reusable housing assembly 106 may be retained on a garment.

In yet other example embodiments, and referring now to FIG. 35, a reservoir assembly 102 may include at least one safety pin 426 coupled thereto. The pin body 428 of the safety pin 426 may be fed through and back out of a garment 416. The pin body 428 may then be engaged with the clasp 430 of the safety pin 426 to retain the reservoir assembly 102 (and any coupled reusable housing assembly 106) in place on a garment 416. The safety pin 426 may include an attachment body 430 (e.g. an elongate plate) which may be coupled to the reservoir assembly 102. The attachment body 430 may be coupled to the reservoir assembly 102 in any suitable manner. For example, the attachment body 430 may be overmolded into, adhered to, retained in place via a swaging operation, press fit, or snap fit into place on the reservoir assembly 102.

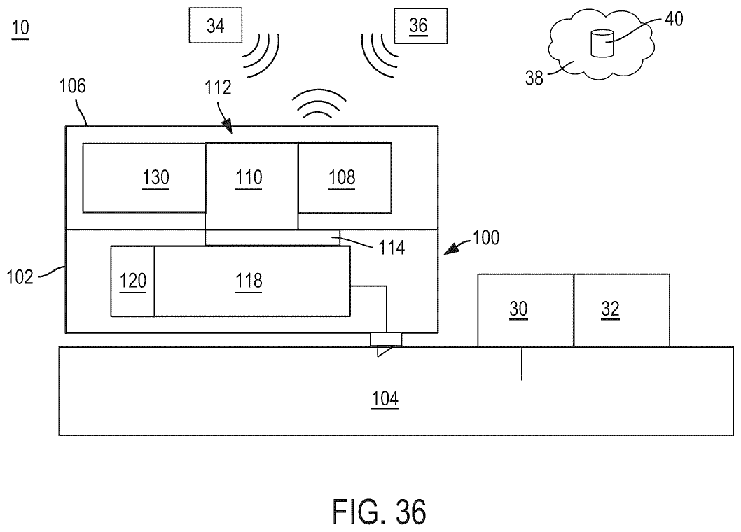

Referring now to FIGS. 36-38, various alternative embodiments of the drug delivery system 10 depicted in FIG. 1 are shown. In each of the example drug delivery systems 10 shown in FIGS. 36-38, the dispensing assembly 100 includes at least one access member 16. Each of the access members 16 may be a puncturing body which may be disposed at least partially within a patient when the dispensing assembly 100 is in use. Where the dispensing assembly 100 is utilized with a socket 324, the socket 324 may include an aperture through which the access members 16 may pass in order to access the patient 104.

Referring primarily to FIG. 36, the drug delivery system 10 may omit an infusion device 186 and the tubing 184 fluidically coupling the infusion device 186 to the dispensing assembly 100. In place of the infusion device 186, one or more access member 16 which is a delivery sharp may be included as part of the dispensing assembly 100. For example, various reservoir assembly 102 embodiments may include one or more delivery sharp type access member 16.

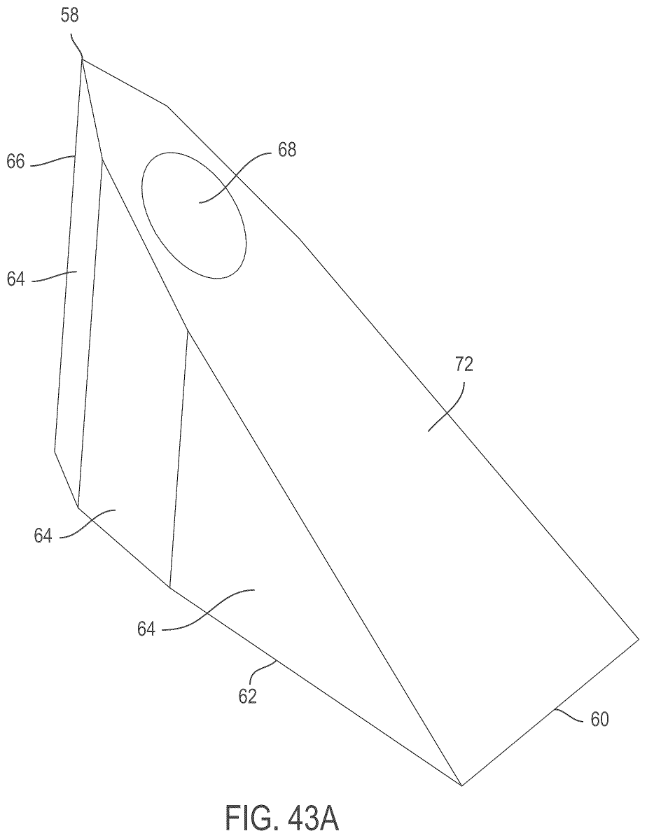

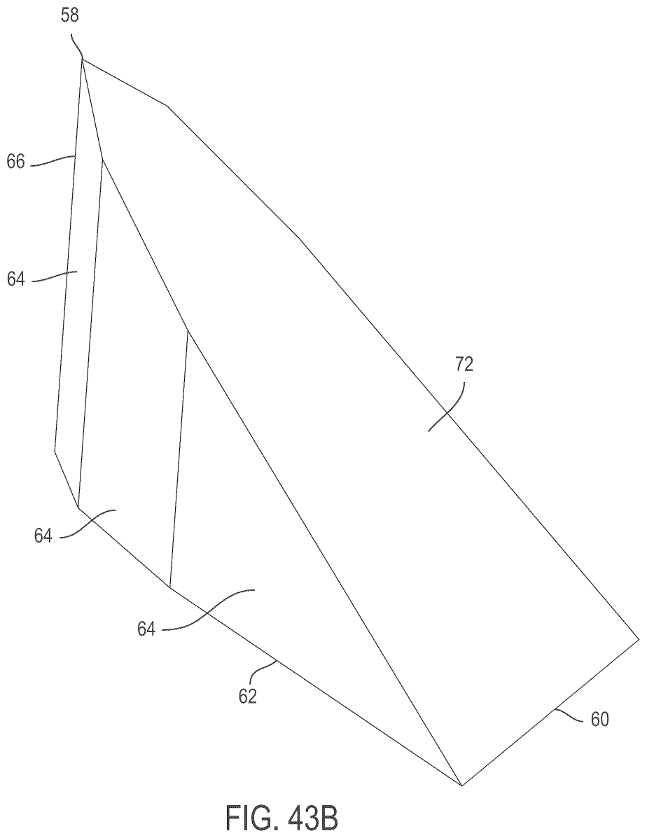

The delivery destination may be a subcutaneous destination (the access member could be a standard gauge needle or a cannula), though in other embodiments may be a shallow delivery destination. Where the delivery destination is a shallow delivery destination, the at least one access member 16 may deliver fluid into a portion of the skin between the stratum corneum and subcutaneous tissue. Shallow delivery destinations may include an epidermal or dermal target location or may, for example, target a junctional area between the epidermis and dermis or dermis and subcutis. The delivery destination may be an intradermal delivery destination. Where the destination is a shallow delivery destination, the delivery sharp type access member(s) 16 may include one or more silicon microneedle(s). In various examples, such microneedles may be included in an array of microneedles all formed from the same silicon wafer. Each microneedle may include a flow lumen 68 extending therethrough which is in fluid communication with the reservoir 118 of the dispensing assembly 100. An example microneedle which may be used is described in relation to FIG. 43A.

Agents supplied may include drugs which are generally supplied as a continuous or substantially continuous infusion though other drugs may also be used. This may include small molecules, biologicals, recombinantly produced pharmaceuticals, and analogs thereof. In various examples, the dispensing assembly 100 may deliver an agent which affects the cardiovascular system or blood vessels. For example, a dispensing assembly 100 may deliver a vasodilator. In certain examples, a drug for the treatment of pulmonary arterial hypertension such as Treprostinil may be delivered. In some examples, a dispensing assembly 100 may deliver a peptide such as a regulatory hormone. In some examples, the agent may be a drug for the treatment of diabetes or a drug which acts to alter blood glucose levels. In certain examples, the dispensing assembly 100 may deliver insulin. In certain embodiments, a dispensing assembly 100 may deliver glucagon. Chemotherapy drugs may also be delivered via the set 12. In some embodiments, multiple agents may be delivered by one or more dispensing assemblies 100 of the system 10. For example, any of the agents described above may be delivered. Where references are made to a specific agent or embodiments are presented in the context of a specific agent it shall be understood that this is merely exemplary. Other agents may be delivered via dispensing assemblies described herein.

Still referring to FIG. 36, in various embodiments, the drug delivery system 10 may also include one or more analyte sensors 30. The analyte sensor(s) 30 may generate data related to a level of an analyte of interest in a patient. The analyte sensor(s) 30 may include an amperometric sensor which generates an electric current which is in proportion to the level of an analyte of interest in the patient. The analyte sensor(s) 30 may include one or more electrode arrangement and may be covered, coated, or otherwise associated with an enzyme specific to the analyte of interest to facilitate such sensing. Any chemistry known for a desired analyte may be used (e.g. glucose oxidase where blood glucose levels are to be monitored). Some examples may include an exclusion membrane may separate the enzyme form the rest of the patient anatomy and my block, for example, large molecules, molecules above a certain molecular weight (e.g. if glucose is the analyte of interest), or interfering substances. Additionally, certain embodiments may include a membrane which may increase biocompatibility. In certain implementations, an analyte sensor 30 may be a blood glucose monitor. The analyte sensor 30 may be separate from the dispensing assembly 100 and may be in communication with the dispensing assembly 100. The analyte sensor(s) 30 may monitor fluid in a subcutaneous space in some examples. In alternative examples, the analyte sensor(s) 30 may monitor patient fluid at a shallow location. The analyte sensor(s) 30 may, for example, monitor fluid in a portion of the skin between the stratum corneum and subcutaneous tissue. The sensing location may be an intradermal location in some examples. Where references are made to a specific analyte or embodiments are presented in the context of a specific analyte it shall be understood that this is merely exemplary. Other analytes may be monitored.

The analyte sensor(s) 30 may be separate from the dispensing assembly 100 and may include or be associated with a transmitter 32. The transmitter 32 may be included within a housing for an analyte sensor 30 or may alternatively be a separate component which may, for example, dock with the sensor 30 and transmit data collected by the analyte sensor 30. The transmitter 32 may be a wireless transmitter such as a radio frequency based transmitter 32. For example, the transmitter 32 may be a near field communication transmitter 32 or may be a Bluetooth transmitter 32. In some embodiments, multiple types of transmitters may be included in a transmitter 32 (e.g. NFC and Bluetooth). Analyte sensor(s) 30 and/or transmitters 32 may include a power source (e.g. coin cell battery) and may include a memory for storing sensor data. Analyte sensor(s) 30 may transmit sensor data via a transmitter 32 upon interrogation by another component of the system 10 or may transmit sensor data based upon a predefined schedule. For example, a transmitter may transmit sensor data after some preset number of minutes (e.g. 1-5 minutes) has elapsed since last transmission. Where transmission is based on a predefined schedule, data may also be transmitted upon interrogation by another component of the system 10. The data transmitted may be data from individual sensor readings or may be numerically processed data from an analyte sensor 30 (e.g. average of sensor readings over some period of time). Individual sensor readings may be taken at a given time or each individual sensor reading may be rendered from integrated sensor signal over some period of time. In certain examples, the transmitter 32 may also transmit alarms in the event that data from an analyte sensor 30 warrants alerting a user of the system 10. These alarms may be transmitted by the transmitter 32 independent of any predefined schedule and/or without the need for any interrogation by another component of the system 10.

Any of the analyte sensors 30 described herein may forego a transmitter 32 and instead have (or establish when data transfer is desired) a wired or other physical connection to another component of the system 10. In embodiments where a transmitter 32 is included, wired connection may still be possible to access data from the sensor 30 if desired.

Sensor data may be transferred to the dispensing assembly 100, a first interface 34 of the system 10, a second interface 36 of the system 10 (third, fourth, fifth, etc. interfaces may also be included in some examples), a database 40 in the cloud 38, or some combination thereof. Additionally, data may be transmitted to one or more of the above and that component may then pass the data to other components of the system 10. In certain examples, the first interface 34 may be a reader for the analyte sensor(s) 30. The reader may be a dedicated reader or may be a smart device such as a smart phone in some embodiments. The second interface 36 may be a smart device. In some examples, the first interface 34 may be a smart phone and the second interface 36 may be a smart watch, tablet, or second smart phone (e.g. that of a parent, guardian, caregiver, etc.). Any of the dispensing assemblies 100, first or second interface 34, 36, or cloud 38 may generate and transmit an alarm to other components of the system 10 should data received meet certain predefined criteria.

The dispensing assembly 100 may receive data from the transmitter 32 (directly or indirectly) and the controller 108 of the dispensing assembly 100 may analyze this data to inform administration of agent from the dispensing assembly 100. Delivery of agent may thus be closed loop in certain examples. Delivery of agent in other examples may be open loop, but data from the analyte sensor(s) 30 may, for example, be used to inform generation of alerts by the controller 108.

The controller 108 of the dispensing assembly 100 may initiate or halt delivery of agent in the event that the analyte level or analyte level trend is determined to be in breach of a threshold or outside of a predefined range. The controller 108 may also adjust delivery of agent in the event that certain criteria are met. For example, the controller 108 may increase or decrease an infusion rate in the event that data from the analyte sensor(s) 30 indicate analyte levels are changing in correspondence with a predefined trend. In some embodiments where the agent serves to decrease analyte levels, if analyte levels are decreasing, the delivery rate may be lowered or delivery may be halted. If analyte levels are increasing, the delivery rate may be increased by the controller 108. The opposite may be true in scenarios where the agent serves to increase the analyte level. In certain embodiments, the dispensing assembly 100 may pump fluid to the delivery sharp type access members 16 at a basal rate and may occasionally provide a bolus to the patient. Depending on the rate of change in the analyte level, the basal delivery rate may be adjusted by the controller 108 or the controller 108 may orchestrate administration of a bolus (or the basal rate may be adjusted in addition to the administration of a bolus). Where multiple agents are delivered (e.g. to different sets of access members 16) by one or more dispensing assembly 100, the type of agent delivered may be altered. For instance, if an agent which decreases analyte levels is being administered and analyte levels are decreasing at greater than a certain rate, delivery may switch to an agent which serves to increase analyte levels (and vice versa).

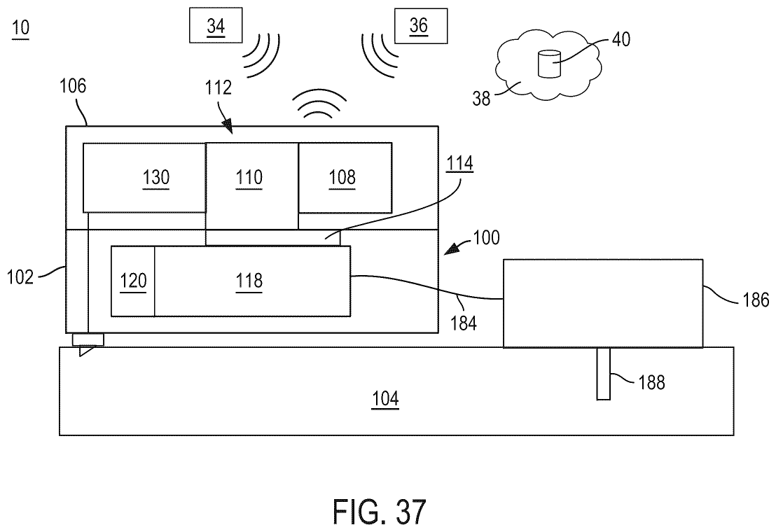

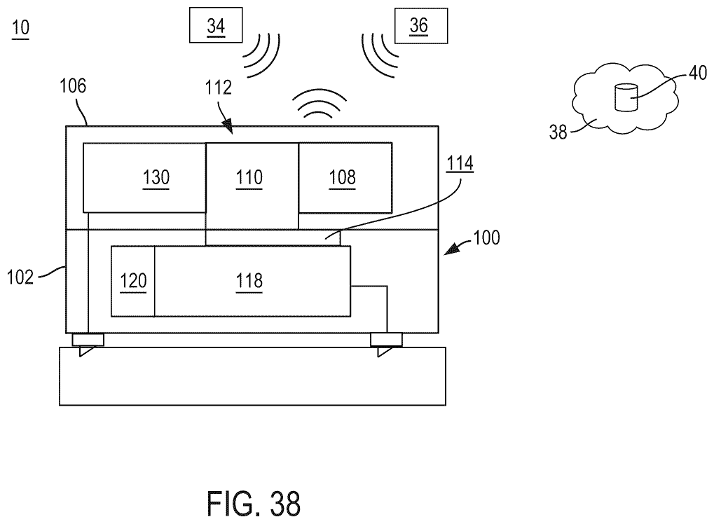

Referring now to FIGS. 37-38, in certain examples, the dispensing assembly 100 may include at least one sensing type access member 16. Sensing type access members 16 may be micropenetrator type access members 16. Micropenetrator type access members 16 may be similar or identical to the delivery sharp type access members 16, however, no lumen extending through the micropenetrator need be present. One such example micropenetrator is described in relation to FIG. 43B. Microneedles may also be referred to herein as micropenetrators, but when used to deliver fluid from a dispensing assembly 100 they would include a lumen as opposed to being solid structures.

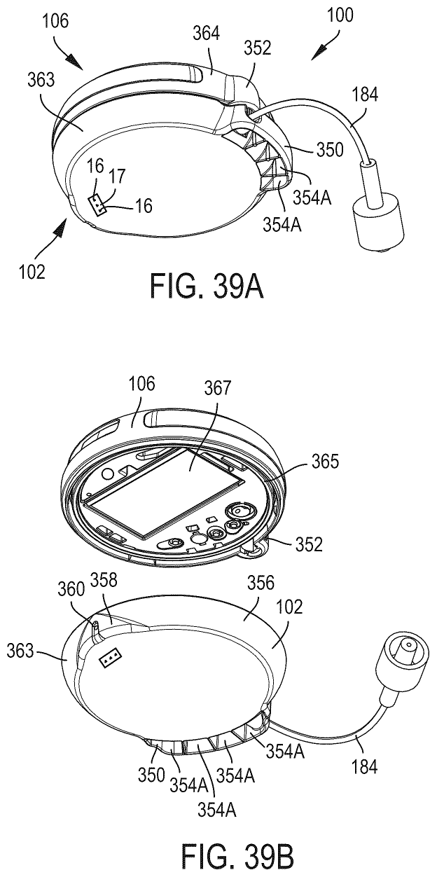

The sensing type access member(s) 16 may for instance be included in a reservoir assembly 102 of a dispensing assembly 100. Electrical connections for the sensing type access member(s) 16 may be included in the reservoir assembly 102. The electrical connections may engage with cooperating electrical connections of the reusable housing assembly 106 when the reservoir assembly 102 is coupled to the reusable housing assembly 106. In embodiments where the reservoir assembly 102 includes a shell 363 (see, e.g. FIG. 39A-39B), engagement of the electrical connections in the reservoir assembly 102 and the reusable housing assembly 106 may be established outside of the environmental seal 365 (see, e.g., FIG. 39B) as the reservoir assembly 102 and reusable housing assembly 106 are coupled. For example, there may be conductive paths on an interior face of the shell 363 which are in electrical communication with the sensing type access member(s) 16. The reusable housing assembly 106 may have conductive pads (or another suitable electrical contact) which are in communication with the controller 108. As the reservoir assembly 102 is coupled to the reusable housing assembly 106, the conductive paths may contact the conductive pads placing the reusable housing assembly 106 into operative engagement with the sensing type access member(s) 16. Alternatively, engagement of the electrical connections in the reservoir assembly 102 and the reusable housing assembly 106 may be established within the environmental seal 365 (see, e.g., FIG. 39B) as the reservoir assembly 102 and reusable housing assembly 106 are coupled. In such embodiments, electrical communication pathways may extend from the sensing type access members 16 and through the cassette. The reusable housing assembly 106 may include conductive contacts or pads which may come into abutment with electrical communication pathways extending through the cassette when the reservoir assembly 102 and reusable housing assembly 106 are coupled.

Including sensing type access members 16 in a dispensing assembly 100 may be desirable for a variety of reasons. For example, the dispensing assembly 100 may include a transmitter 130. Thus a separate transmitter 32 which may be associated with an analyte sensor 30 may be omitted. Additionally, any processing hardware which may be associated with a separate analyte sensor 30 and/or transmitter 32 may be omitted. Instead, the controller 108 in the reusable housing assembly 106 may be used. Additionally a separate power source may be omitted. Any power needed to make use of the sensing type access members 16 may be drawn from a battery 367 (see, e.g., FIG. 39B) of the reusable housing assembly 106. Additionally, communication dropout issues may be less of a concern.

In some embodiments, a dispensing assembly 100 including at least one sensing type access member 16 may be coupled (e.g. via tubing 184) to a separate infusion device 186 which may provide access to a delivery destination in a patient 104 (see, e.g., FIG. 37). In alternative embodiments, all of the patient access components of the drug delivery system 10 may be included in the dispensing assembly 100 (see, e.g., FIG. 38). Thus a dispensing assembly 100 may include delivery sharp type access members 16 as well as sensing type access members 16. In still other embodiments, a dispensing assembly 100 may have delivery sharp type access members 16 and sensing type access members 16 and the drug delivery system 10 may have at least one of a separate infusion device 186 and/or analyte sensor 30.

Referring now primarily to FIGS. 39A-39B, a perspective view and an exploded perspective view of a dispensing assembly are respectively depicted. As shown, the example reservoir assembly 102 includes a shell 363, however, in other embodiments, only the cassette portion of a reservoir assembly 102 may be present. The example dispensing assembly 100 includes a set of access members 16. The access members 16 may be delivery type access member 16 or sensing type access members 16. Both types of access members 16 may also be included in the example embodiment. The example access members 16 are depicted as micropenetrators. The example access members 16 are included on a sharp bearing body 17 which may be integral with the micropenetrators. Where the micropenetrators are sensing type access members 16, there may be two sharp bearing bodies 17 each bearing at least one micropenetrator such that electrical isolation between different micropenetrators may be maintained.

The sharp bearing body 17 may be coupled to the reservoir assembly 102 (shell 363 or cassette) during an injection molding operation in certain examples. In alternative examples, the sharp bearing body 17 may be coupled to the reservoir assembly 102 (shell 363 or cassette) via adhesive. In some examples including a shell 363, the micropenetrators may protrude through a passage in the shell 363 and could, for example, be coupled to the cassette. The exterior surface of the reservoir assembly 102 surrounding the sharp bearing body 17 may be raised with the micropenetrators extending proud of the raised surface. Thus, the micropenetrators may be disposed on or extend through a protuberance of the reservoir assembly 102 which may assist in keeping the micropenetrators robustly in place within the patient 104.

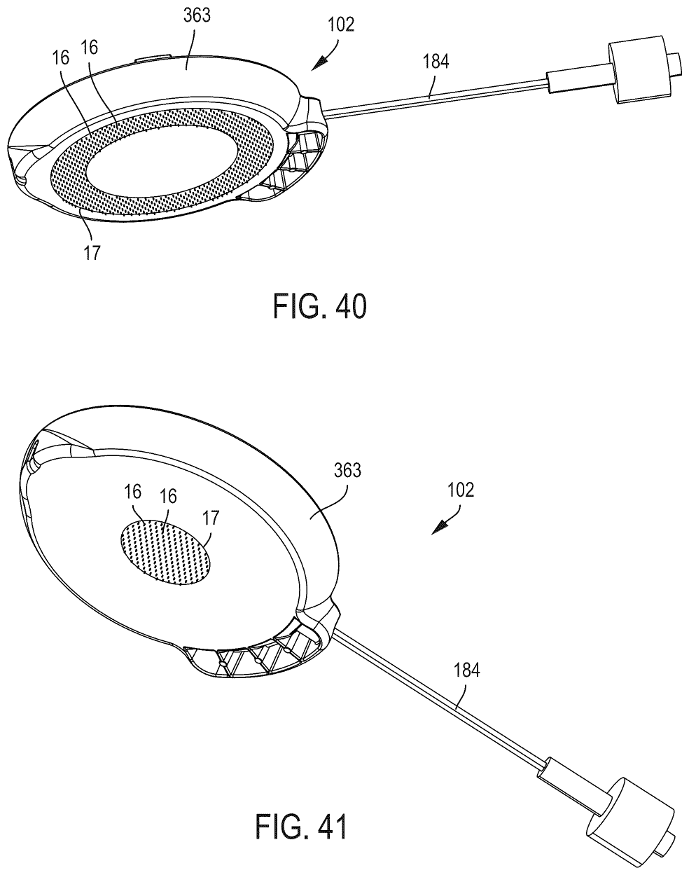

Referring now to FIGS. 40-41, perspective views of two exemplary reservoir assemblies 102 are depicted. Sharp bearing bodies 17 may come in any suitable size and shape and may include any desired number of access members 16. FIG. 41 and FIG. 40 respectively depict a round and a ring shaped sharp bearing body 17. The access members 16 shown are disposed spaced at regular intervals on the sharp bearing body 17 though need not be in all embodiments. Any placement pattern or spacing for the access members 16 may be used. The shapes of the sharp bearing bodies 17 and number of access members 16 thereon are merely illustrative. Additionally, multiple sharp bearing bodies 17 each having one or more access members 16 defined thereon may be included. In such examples, the sharp bearing bodies 17 may be disposed in any desired arrangement. Where multiple sharp bearing bodies 17 are included, the type of access member 16 may differ from sharp bearing body 17 to sharp bearing body 17.

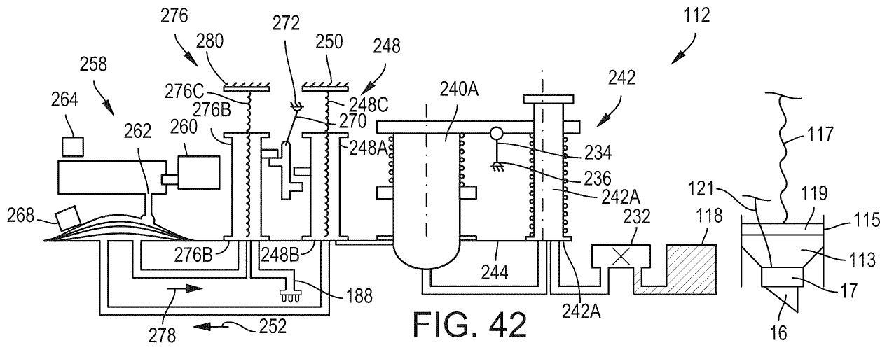

Referring now primarily to FIG. 42, an alternative embodiment of the actuation assembly 112 depicted in FIG. 3A is shown. The actuation assembly 112 is the same as that depicted in FIG. 3A, however, the cannula 188 has been replaced with a set of microneedles on a sharp bearing body 17. As mentioned elsewhere herein, the microneedles may be included in the dispensing assembly 100 (e.g. reservoir assembly 102) and may be in fluid communication with the fluid output for the actuation assembly 112. A carriage 113 with a sensing micropenetrator extending from a sharp bearing body 17 is also depicted in FIG. 42. Though shown with a single micropenetrator, there may be multiple sensing micropenetrators coupled to the carriage 113 (e.g. two electrically isolated micropenetrators). The carriage 113 may be disposed in a guide channel 115 defined in a part of the dispensing assembly 100. For example the carriage 113 may be disposed in a guide channel 115 of the reservoir assembly 102. An electrical communication path 121 extends from the sharp bearing body 17 and may be engaged with components in a reusable housing assembly 106 when the reservoir assembly 102 and reusable housing assembly 106 are coupled as described elsewhere herein. At least one bias member 117 may be included and may exert a force which tends to drive the carriage 113 in the direction of the patient 104. This may help to ensure that a sensing micropenetrator is robustly held in engagement with the sensing location in the patient 104. In some embodiments a plunger 119 or the like may be disposed intermediate the carriage 113 and the bias member 117. The plunger 119 and bias member 117 may be included in the reusable housing assembly 106. When the reservoir assembly 102 and the reusable housing assembly 106 are initially advanced against one another for coupling, the plunger 119 may be pressed against a surface of the reservoir assembly 102. This may cause the plunger 119 to retract into the reusable housing assembly 106 and the bias member 117 may distort in response. The reservoir assembly 102 (or reusable housing assembly 106) may be rotated to couple the reservoir assembly 102 and reusable housing assembly 106 to one another. In the coupled state, the plunger 119 may pass into alignment with the guide channel 113. The bias member 117 may automatically restore to a less distorted state and the plunger 119 may exert pressure upon the carriage 113 which may tend to hold the sensing micropenetrator in place within the patient 104.

Referring now also to FIG. 43A, where microneedles are used, the microneedles described herein may, in certain embodiments, be MEMS produced, polyhedral (e.g. pyramidal), silicon crystal microneedles. These microneedles may be no greater than 1 mm in height, e.g. 0.6 mm or 0.8 mm (though longer or shorter microneedles may also be used). In some embodiments, microneedles may be 1200-1500 microns in height or perhaps longer in some examples. In some embodiments, microneedles may have a height sufficient to puncture at least some distance into subcutaneous tissue. At least some edges of the microneedles may be rounded or filleted, though such microneedles may still be referred to herein as polyhedral. In some examples and as shown in FIG. 43A, the microneedles described herein may be generally in the shape of a heptagonal prism (though pentagonal, nonagonal, and other polygonal prisms may also be used as the base shape) which has been diagonally sected to form a heptagonal ramp or pointed wedge. In such embodiments, the heptagonal prism may be sected by a plane extending from a vertex 58 of the top face of the prism through the most distal side 60 of the base 62. At least two sides of the base of the microneedle may be parallel. The side walls 64 may extend substantially perpendicularly from the base 62. A microneedle may be substantially symmetric about a line of symmetry extending from the vertex 58 to a point above the center of the most distal side 60. In other embodiments, a microneedle may be conically shaped. Any other suitable shape may be used. In the example, the vertex 58 is shown as a point which forms a tip of the microneedle. In other embodiments, this portion of a microneedle may be rounded (though may still be referred to herein as a vertex 58 and such microneedles may still be referred to as pointed). In such embodiments, the back facing edge 66 may be a round face or the back facing edge 66 and the adjacent side walls 64 may be replaced by a rounded face.

The points or tips of microneedles described herein may be solid and the flow lumens 68 through the microneedles may be offset from the points or tips (in FIG. 43A the vertex 58 forms the tip) of the microneedles. Hollow tipped microneedles in which the flow lumen 68 extends to the tip of the microneedle may also be utilized. In some embodiments, the microneedles may be NanoPass hollow microneedles available from NanoPass Technologies Ltd. of 3 Golda Meir, Nes Ziona, Israel. Microneedles may be provided in an array of any suitable number of row and columns on a sharp bearing body 17. The microneedles may be integrally formed with the sharp bearing body 17.

Referring now to FIG. 43B, an example sensing micropenetrator is depicted. Such micropenetrators may be similar to the microneedle embodiments described above in relation to FIG. 43A. Sensing micropenetrators may, however, be lumenless as shown. Each of the sensing micropenetrators may be constructed of etched silicon. Each of the micropenetrators may be disposed on its own sharp bearing body 17 such that they are out of direct electrical communication with one another. Sensing micropenetrators may be provided in pairs. One sharp bearing body 17 and micropenetrator may serve as a counter/reference electrode while the other sharp bearing body 17 and micropenetrator of the pair may serve as a sensing electrode. Conductive traces which may enter into communication with cooperative conductive paths in the reusable housing assembly 106 may be associated with each sharp bearing body of the pair. Any suitable analyte sensing chemistry or arrangement from any manufacturer may be incorporated on the sensing electrode. A glucose sensing arrangement may be used. For example, a glucose oxidase chemistry could be used to sense glucose levels in body fluid.

Various alternatives and modifications can be devised by those skilled in the art without departing from the disclosure. Accordingly, the present disclosure is intended to embrace all such alternatives, modifications and variances. Additionally, while several embodiments of the present disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. And, those skilled in the art will envision other modifications within the scope and spirit of the claims appended hereto. Other elements, steps, methods and techniques that are insubstantially different from those described above and/or in the appended claims are also intended to be within the scope of the disclosure.

The embodiments shown in drawings are presented only to demonstrate certain examples of the disclosure. And, the drawings described are only illustrative and are non-limiting. In the drawings, for illustrative purposes, the size of some of the elements may be exaggerated and not drawn to a particular scale. Additionally, elements shown within the drawings that have the same numbers may be identical elements or may be similar elements, depending on the context.

Where the term “comprising” is used in the present description and claims, it does not exclude other elements or steps. Where an indefinite or definite article is used when referring to a singular noun, e.g. “a” “an” or “the”, this includes a plural of that noun unless something otherwise is specifically stated. Hence, the term “comprising” should not be interpreted as being restricted to the items listed thereafter; it does not exclude other elements or steps, and so the scope of the expression “a device comprising items A and B” should not be limited to devices consisting only of components A and B.

Furthermore, the terms “first”, “second”, “third” and the like, whether used in the description or in the claims, are provided for distinguishing between similar elements and not necessarily for describing a sequential or chronological order. It is to be understood that the terms so used are interchangeable under appropriate circumstances (unless clearly disclosed otherwise) and that the embodiments of the disclosure described herein are capable of operation in other sequences and/or arrangements than are described or illustrated herein.

What is claimed is:

1. A retention assembly for an ambulatory infusion pump assembly comprising:
an adhesive bearing pad including a main region and a number of petal shaped protrusions extending from the periphery of the main region and being spaced about the main region at regular angular intervals; and
a socket coupled to the adhesive pad comprising:
a base portion with a centrally disposed socket aperture;
a wall extending from a periphery of the base portion and having at least one wall ledge extending from a portion of the wall most distal the base portion, the wall interrupted by a respective fenestration aligned with each of the at least one wall ledge;
an appendage projecting from the base portion;
a clip at an end of the appendage most distal to the base portion, the clip having a raised wall with at least one clip ledge extending from the raised wall, the clip ledge being positioned intermediate the appendage and the end of the raised wall most distal the appendage, there being a respective passage through the appendage associated with each clip ledge, each clip ledge extending over the respective passage; and
at least one alignment projection extending from the appendage.

2. The retention assembly of claim 1, wherein the socket further comprises a centrally disposed socket aperture.

3. The retention assembly of claim 2, wherein the adhesive bearing pad includes a pad aperture aligned with the socket aperture.

4. The retention assembly of claim 1, wherein the base portion includes at least one magnet.

5. The retention assembly of claim 1, wherein the base portion includes at least one retention post.

6. The retention assembly of claim 1, wherein the base portion further comprises at least one retention post clutch.

7. The retention assembly of claim 1, wherein the retention assembly further comprises a release liner having a peripheral protuberance.

8. The retention assembly of claim 1, wherein the base portion and appendage are formed of rigid material.

9. The retention assembly of claim 8, wherein the socket further comprises a compliant portion overmolded onto at least the base portion.

10. The retention assembly of claim 1, wherein the at least one alignment projection includes at least one tooth projection.

11. The retention assembly of claim 1, wherein the at least one alignment projection is tapered such that its cross-sectional area decreases as distance from the base portion increases.

12. The retention assembly of claim 1, wherein the wall is present along a minority of the periphery of the base portion.

13. The retention assembly of claim 1, wherein the wall is present along no more than a third of the periphery of the base portion.

14. The retention assembly of claim 1, wherein the adhesive bearing pad is coupled to the socket by one of a heat stake and a sonic weld.

15. The retention assembly of claim 1, wherein the socket includes at least one air flow channel recessed into a face of the base portion.

16. The retention assembly of claim 1, wherein the base portion includes a least one pass-through extending therethrough and wherein each of the at least one pass-through is aligned with a respective aperture in the adhesive pad.

17. An ambulatory infusion system comprising:
An infusion pump assembly comprising;
a first portion having a controller, a transmitter, and a mechanical actuation assembly;
a second portion including a reservoir and a fluid path extending from the reservoir toward a delivery outlet;
a securement body at least partially formed from a material selected from a list consisting of a ferromagnetic material and permanently magnetized material; and
a retainer body separate from the infusion pump including at least one magnet configured to magnetically hold the securement body in place when a gap of at least one millimeter which is filled with garment material is present between the retainer body and securement body.

18. The system of claim 17, wherein the mechanical actuation assembly includes no motor.

19. The system of claim 17, wherein the mechanical actuation assembly includes at least one shape memory actuator.

20. A reservoir assembly for an ambulatory infusion pump comprising:
a first unit comprising:
a reservoir with a main interior volume;
a septum having a face in fluid communication with the main interior volume;
a pump chamber, set of valve chambers, and a variable volume sensing chamber in fluid communication with each other and the main interior volume via a set of flow paths;
at least one flexible membrane covering the pump chamber, set of valve chambers, and the variable volume sensing chamber;
a length of tubing coupled to an outlet of the set of flow paths;
a shell unit including a tab and at least one shell recess, the first unit being coupled within the shell unit; and
at least one ambulatory mounting component selected from a list consisting of at least one magnet, at least one ferromagnetic body, at least one retention post, and at least one retention post clutch.
